(19) United States
(12) Patent Application Publication
(10) Pub. No.: US 2025/0101043 A1
(43) Pub. Date: Mar. 27, 2025

(54) KRAS INHIBITORS

(71) Applicant: Eli Lilly and Company, Indianapolis, IN (US)

(72) Inventors: Mario BARBERIS, Madrid (ES); Santiago CARBALLARES MARTIN, Madrid (ES); Victoriano Molero FLÓREZ, Madrid (ES); Deqi GUO, Carmel, IN (US); Richard Duane JOHNSTON, Greenfield, IN (US); Isabel ROJO GARCIA, Madrid (ES); Gaiying ZHAO, Carmel, IN (US)

(21) Appl. No.: 18/897,646

(22) Filed: Sep. 26, 2024

(30) Foreign Application Priority Data
Sep. 27, 2023 (EP) 23382983.7

(51) Int. Cl. C07D 519/00; A61K 31/517; A61K 45/06

(52) U.S. Cl. CPC C07D 519/00; A61K 31/517; A61K 45/06

(57) ABSTRACT

The present invention provides compounds of the formula:

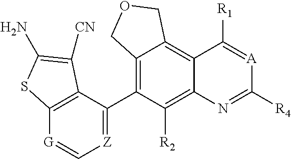

wherein A, Z, G, R₁, R₂, and R₄ are as described herein, pharmaceutically acceptable salts thereof, and methods of using these compounds and pharmaceutically acceptable salts thereof for treating patients for cancer.

### BACKGROUND

The MAPK/ERK signaling pathway relays extracellular stimuli to the nucleus, thereby regulating diverse cellular responses including cell proliferation, differentiation, and apoptosis. KRas protein is an initiator of the MAPK/ERK signaling pathway and functions as a switch responsible for inducing cell division. In its inactive state, KRas binds guanosine diphosphate (GDP), effectively sending a negative signal to suppress cell division. In response to an extracellular signal, KRas is allosterically activated allowing for nucleotide exchange of GDP for guanosine triphosphate (GTP). In its GTP-bound active state, KRas recruits and activates proteins necessary for the propagation of growth factor induced signaling, as well as other cell signaling receptors. Examples of the proteins recruited by KRas-GTP are c-Raf and PI3-kinase. KRas, as a GTP-ase, converts the bound GTP back to GDP, thereby returning itself to an inactive state, and again propagating signals to suppress cell division. KRas gain of function mutations exhibit an increased degree of GTP binding and a decreased ability to convert GTP into GDP. The result is an increased MAPK/ERK signal which promotes cancerous cell growth. Missense mutations of KRas at codon 12 are the most common mutations and markedly diminish GTPase activity.

Oncogenic KRas mutations have been identified in approximately 30% of human cancers and have been demonstrated to activate multiple downstream signaling pathways. Despite the prevalence of KRas mutations, it has been a difficult therapeutic target. (Cox, A. D. Drugging the Undruggable RAS: Mission Possible? Nat. Rev. Drug Disc. 2014, 13, 828-851; Pylayeva-Gupta, y et al. RAS Oncogenes: Weaving a Tumorigenic Web. Nat. Rev. Cancer 2011, 11, 761-774).

Thus far, work has focused on KRas G12C mutant inhibitors (e.g., WO2019/099524, WO2020/081282, WO2020/101736, WO2020/146613, and WO2021/118877 disclose KRas G12C inhibitors), whereas WO2021/041671 discloses small molecules inhibitors of KRas G12D and WO2017/011920 discloses small molecule inhibitors of KRas G12C, G12D, and G12V.

There remains a need to provide alternative, small molecule KRas inhibitors. In particular, there is a need to provide more potent, orally deliverable KRas inhibitors that are useful for treating cancer. More particularly, there is a need to provide small molecule inhibitors that specifically inhibit KRas GTP activity. Further, there is a desire to provide KRas inhibitors that exhibit enhanced pharmacokinetic/pharmacodynamic properties. Also, there is a need to provide more potent KRas inhibitors that exhibit increased efficacy with reduced or minimized untoward or undesired effects. Further, there is a need to provide more potent KRas inhibitors that exhibit selective inhibition preference for KRas G12D mutant over KRas wild-type. The present invention addresses one or more of these needs by providing novel KRas inhibitors.

### SUMMARY

Compounds of Formula I are provided herein:

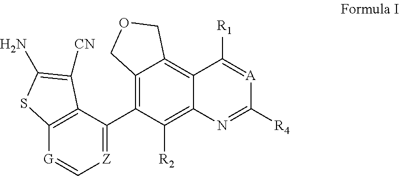

wherein
A is —C(H)— or —N—;
Z is —C(R_{3c})— or —N—;
G is —C(R_{3b})— or —N—;
R₁ is —H, or a group of the formula

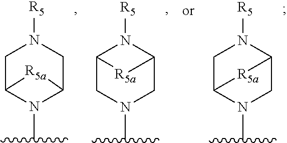

R₂ is —H, halogen, or methyl;
R_{3b}, and R_{3c} are each independently —H, halogen, or methyl;
R₄ is a group of the formula

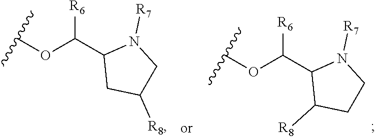

R₅ is a C₁₋₄ alkyl optionally substituted with one or more hydroxyl, or methoxy;
R₅ₐ is C₁₋₃ alkylene;
R₆ is C₁₋₃ alkyl;
R₇ is —H, or C₁₋₃ alkyl; and
R₈ is —H, halogen, or C₁₋₃ alkoxy; or a pharmaceutically acceptable salt thereof.

Also provided herein are methods of using the compounds of Formula I, pharmaceutically acceptable salts thereof, and pharmaceutical compositions thereof, to treat cancer, in particular for the treatment of lung cancer, pancreatic cancer, cervical cancer, esophageal cancer, endometrial cancer, ovarian cancer, cholangiocarcinoma, and colorectal cancer. The methods include administering a therapeutically effective amount of a compound of Formula I, or a pharmaceutically acceptable salt thereof, to a patient in need thereof.

Further provided herein, are compounds of Formula I, and pharmaceutically acceptable salts thereof, for use in therapy. Additionally provided herein, are the compounds of Formula I, and pharmaceutically acceptable salts thereof, for use in the treatment of cancer, in particular for the treatment of lung cancer, pancreatic cancer, cervical cancer, esophageal cancer, endometrial cancer, ovarian cancer, cholangiocarcinoma, and colorectal cancer. Also additionally provided herein is the use of compounds of Formula I, or pharmaceutically acceptable salts thereof, in the manufacture of a medicament for treating cancer, in particular for the treatment of lung cancer, pancreatic cancer, cervical cancer, esophageal cancer, endometrial cancer, ovarian cancer, cholangiocarcinoma, and colorectal cancer.

### DETAILED DESCRIPTION

Novel inhibitors of the KRas gain of function mutation G12D are described herein. These new compounds could address the needs noted above for inhibitors of KRas GTP activity in gain of function mutants in the treatment of cancers such as lung cancer, colorectal cancer, pancreatic cancer, bladder cancer, cervical cancer, endometrial cancer, ovarian cancer, cholangiocarcinoma or esophageal cancer. Some of these new KRas inhibitor compounds are selective to KRas G12D mutants over wild-type KRas. Additionally, some of these new KRas inhibitor compounds are non-selective and inhibit both wild-type KRas and KRas G12D mutants (and/or possibly other mutant types such as G12C or G12V).

The present invention provides a compound of Formula I:

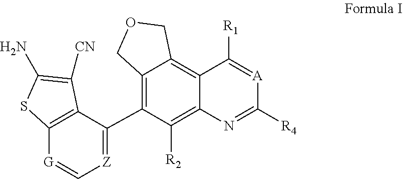

wherein A, G, Z, R₁, R₂, and R₄ are as defined above, or a pharmaceutically acceptable salt thereof.

As used herein, the term halogen means fluoro (F), chloro (Cl), bromo (Br), or iodo (I). As used herein, the term alkyl means saturated linear or branched-chain monovalent hydrocarbon radicals of one to a specified number of carbon atoms, e.g., “C₁₋₄ alkyl” or “C₁₋₃ alkyl.” Examples of alkyls include, but are not limited to, methyl, ethyl, propyl, 1-propyl, isopropyl, butyl, and iso-butyl. As used herein, the term alkylene means saturated linear or branched-chain bivalent hydrocarbon radicals of one to a specified number of carbon atoms, e.g., “C₁₋₃ alkylene.” Examples of alkylenes include, but are not limited to, methylene, ethylene, propylene, 1-propylene, and isopropylene. Examples of C₁₋₃ alkoxy groups include, but are not limited to, methoxy, ethoxy, propoxy, 1-propoxy, and isopropoxy.

The following are further numbered aspects of the invention:
1. A compound of the formula:

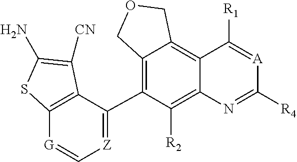

wherein
A is —C(H)— or —N—;
Z is —C(R_{3c})— or —N—;
G is —C(R_{3b})— or —N—;
R₁ is —H, or a group of the formula

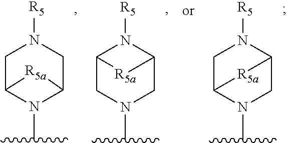

R₂ is —H, halogen, or methyl;
R_{3b}, and R_{3c} are each independently —H, halogen, or methyl;
R₄ is a group of the formula

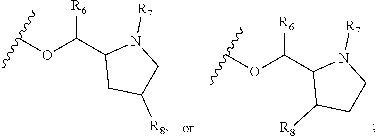

R₅ is a C₁₋₄ alkyl optionally substituted with one or more hydroxyl, or methoxy;
R₅ₐ is C₁₋₃ alkylene;
R₆ is C₁₋₃ alkyl;
R₇ is —H, or C₁₋₃ alkyl; and
R₈ is —H, halogen, or C₁₋₃ alkoxy; or a pharmaceutically acceptable salt thereof.
2. The compound as defined in aspect 1, or pharmaceutically acceptable salt thereof, wherein G is —N—.
3. The compound as defined in aspect 1, or pharmaceutically acceptable salt thereof, wherein G is —C(R3b)-.
4. The compound as defined in aspect 3, or pharmaceutically acceptable salt thereof, wherein G is —C(R3b)-, and R3b is —H, or halogen.
5. The compound as defined in aspect 4, or pharmaceutically acceptable salt thereof, wherein G is —C(F)—.
6. The compound as defined in aspect 4, or pharmaceutically acceptable salt thereof, wherein G is —C(Cl)—.
7. The compound as defined in aspect 4, or pharmaceutically acceptable salt thereof, wherein G is —C(H)—.
8. The compound as defined in aspect 3, or pharmaceutically acceptable salt thereof, wherein G is —C(CH3)-.
9. The compound as defined in any one of aspects 1-8, or pharmaceutically acceptable salt thereof, wherein Z is —N—.
10. The compound as defined in any one of aspects 1-8, or pharmaceutically acceptable salt thereof, wherein Z is —C(R_{3c})—.
11. The compound as defined in aspect any one of aspects 1-8, or pharmaceutically acceptable salt thereof, wherein Z is —C(R_{3c})—, wherein R_{3c} is —H, or —F.
12. The compound as defined in aspect 11, or pharmaceutically acceptable salt thereof, wherein Z is —C(H)—.
13. The compound as defined in aspect 11, or pharmaceutically acceptable salt thereof, wherein Z is —C(F)—.
14. The compound as defined in aspect 1, or pharmaceutically acceptable salt thereof, wherein G is —N—, Z is —C(R_{3c})—, and R_{3c} is —H, or halogen.
15. The compound as defined in aspect 1, or pharmaceutically acceptable salt thereof, wherein G is —N—, and Z is —C(H)—.
16. The compound as defined in aspect 1, or pharmaceutically acceptable salt thereof, wherein G is —N—, and Z is —C(F)—.
17. The compound as defined in aspect 1, or pharmaceutically acceptable salt thereof, wherein G is —C(R_{3b})—, and Z is —N—.
18. The compound as defined in aspect 1, or pharmaceutically acceptable salt thereof, wherein G is —C(R_{3b})—, R_{3b} is —H, or halogen, and Z is —N—.
19. The compound as defined in aspect 1, or pharmaceutically acceptable salt thereof, wherein G is —C(F)—, and Z is —N—.
20. The compound as defined in aspect 1, or pharmaceutically acceptable salt thereof, wherein G is —C(H)—, and Z is —N—.
21. The compound as defined in aspect 1, or pharmaceutically acceptable salt thereof, wherein G is —C(CH₃)—, and Z is —N—.
22. The compound as defined in any one of aspects 1, 3, or 10, or pharmaceutically acceptable salt thereof, wherein R_{3b}, and R_{3c} are each independently —H, or halogen.
23. The compound as defined in any one of aspects 1-22, or pharmaceutically acceptable salt thereof, wherein A is —N—.
24. The compound as defined in any one of aspects 1-22, or pharmaceutically acceptable salt thereof, wherein A is —C(H)—.
25. The compound as defined in any one of aspects 1-24, or pharmaceutically acceptable salt thereof, wherein R₂ is F or Cl.
26. The compound as defined in any one of aspects 1-24, or pharmaceutically acceptable salt thereof, wherein R₂ is F.
27. The compound as defined in any one of aspects 1-24, or pharmaceutically acceptable salt thereof, wherein R₂ is Cl.
28. The compound as defined in any one of aspects 1-27, or pharmaceutically acceptable salt thereof, wherein R₁ is —H.
29. The compound as defined in any one of aspects 1-27, or pharmaceutically acceptable salt thereof, wherein R₁ is a group of the formula

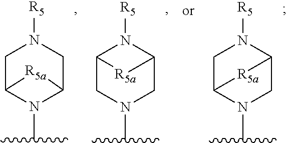

30. The compound as defined in aspect 29, or pharmaceutically acceptable salt thereof, wherein R₁ is a group of the formula

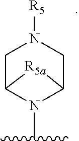

31. The compound as defined in aspect 29 or 30, or pharmaceutically acceptable salt thereof, wherein R₅ₐ is ethylene.
32. The compound as defined in aspect 31, or pharmaceutically acceptable salt thereof, wherein R₁ is selected from

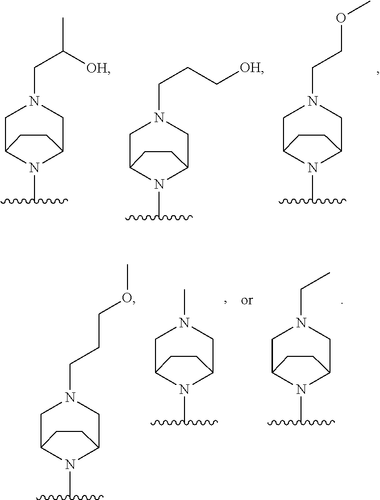

33. The compound as defined in aspect 32, or pharmaceutically acceptable salt thereof, wherein R₁ is selected from

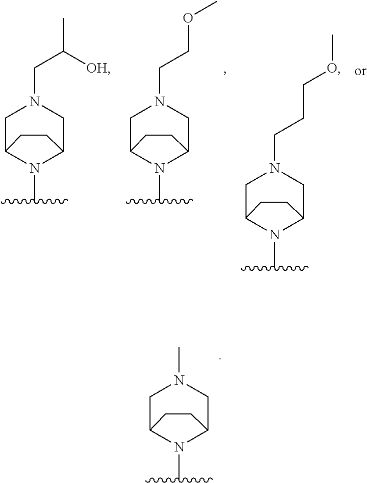

34. The compound as defined in any one of aspects 1-33, or pharmaceutically acceptable salt thereof, wherein R₄ is a group of the formula

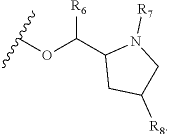

35. The compound as defined in aspect 34, or pharmaceutically acceptable salt thereof, wherein R₄ is a group of the formula

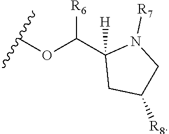

36. The compound as defined in aspect 34, or pharmaceutically acceptable salt thereof, wherein R₄ is a group of the formula

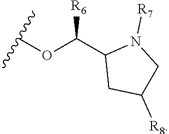

37. The compound as defined in aspect 35 or 36, or pharmaceutically acceptable salt thereof, wherein R₄ is a group of the formula

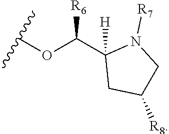

38. The compound as defined in aspect 34, or pharmaceutically acceptable salt thereof, wherein R₄ is a group of the formula

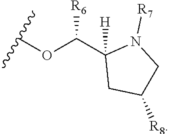

39. The compound as defined in aspect 35 or 38, or pharmaceutically acceptable salt thereof, wherein R₄ is a group of the formula

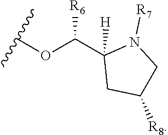

40. The compound as defined in aspect 34, or pharmaceutically acceptable salt thereof, wherein R₄ is a group of the formula

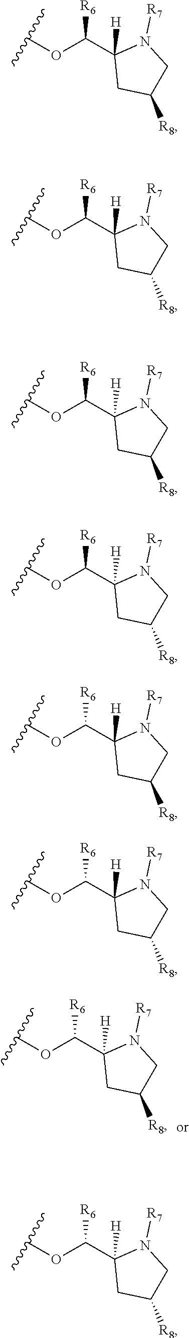

41. The compound as defined in aspect 34, or pharmaceutically acceptable salt thereof, wherein R₄ is a group of the formula

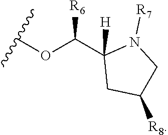

42. The compound as defined in aspect 34, or pharmaceutically acceptable salt thereof, wherein R₄ is a group of the formula

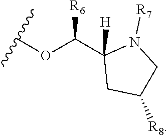

43. The compound as defined in aspect 34, or pharmaceutically acceptable salt thereof, wherein R₄ is a group of the formula

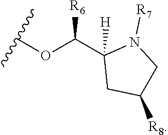

44. The compound as defined in aspect 34, or pharmaceutically acceptable salt thereof, wherein R₄ is a group of the formula

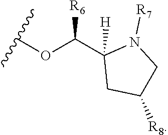

45. The compound as defined in aspect 34, or pharmaceutically acceptable salt thereof, wherein R₄ is a group of the formula

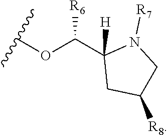

46. The compound as defined in aspect 34, or pharmaceutically acceptable salt thereof, wherein R₄ is a group of the formula

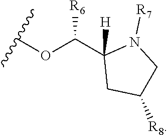

47. The compound as defined in aspect 34, or pharmaceutically acceptable salt thereof, wherein R₄ is a group of the formula R₆H R

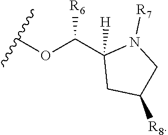

48. The compound as defined in aspect 34, or pharmaceutically acceptable salt thereof, wherein R₄ is a group of the formula

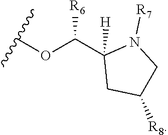

49. The compound as defined in any one of aspects 1-33, or pharmaceutically acceptable salt thereof, wherein R₄ is a group of the formula

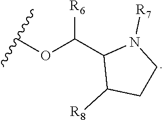

50. The compound as defined in aspect 49, or pharmaceutically acceptable salt thereof, wherein R₄ is a group of the formula

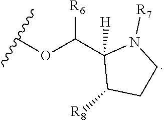

51. The compound as defined in aspect 49, or pharmaceutically acceptable salt thereof, wherein R₄ is a group of the formula

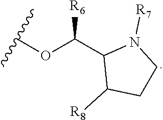

52. The compound as defined in aspect 50 or 51, or pharmaceutically acceptable salt thereof, wherein R₄ is a group of the formula

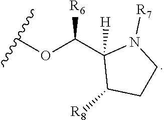

53. The compound as defined in aspect 49, or pharmaceutically acceptable salt thereof, wherein R₄ is a group of the formula

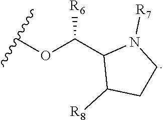

54. The compound as defined in aspect 50 or 53, or pharmaceutically acceptable salt thereof, wherein R₄ is a group of the formula

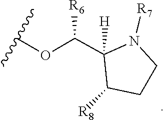

55. The compound as defined in aspect 49, or pharmaceutically acceptable salt thereof, wherein R₄ is a group of the formula

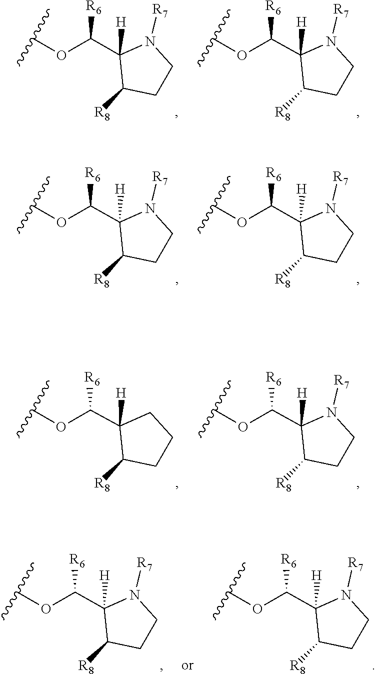

56. The compound as defined in aspect 49, or pharmaceutically acceptable salt thereof, wherein R₄ is a group of the formula

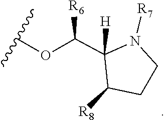

57. The compound as defined in aspect 49, or pharmaceutically acceptable salt thereof, wherein R₄ is a group of the formula

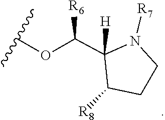

58. The compound as defined in aspect 49, or pharmaceutically acceptable salt thereof, wherein R₄ is a group of the formula

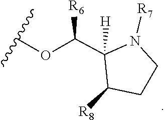

59. The compound as defined in aspect 49, or pharmaceutically acceptable salt thereof, wherein R₄ is a group of the formula

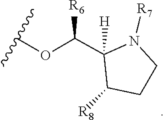

60. The compound as defined in aspect 49, or pharmaceutically acceptable salt thereof, wherein R₄ is a group of the formula

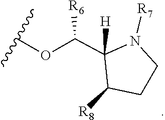

61. The compound as defined in aspect 49, or pharmaceutically acceptable salt thereof, wherein R₄ is a group of the formula

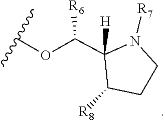

62. The compound as defined in aspect 49, or pharmaceutically acceptable salt thereof, wherein R₄ is a group of the formula

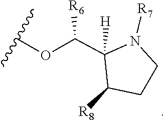

63. The compound as defined in aspect 49, or pharmaceutically acceptable salt thereof, wherein R₄ is a group of the formula

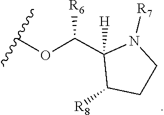

64. The compound as defined in any one of aspects 1-63, or pharmaceutically acceptable salt thereof, wherein R₆ is methyl.
65. The compound as defined in any one of aspects 1-64, or pharmaceutically acceptable salt thereof, wherein R₇ is methyl.
66. The compound as defined in any one of aspects 1-65, or pharmaceutically acceptable salt thereof, wherein R₆, and R₇ are methyl.
67. The compound as defined in any one of aspects 1-66, or pharmaceutically acceptable salt thereof, wherein R₈ is —H, —F, or methoxy.
68. The compound as defined in aspect 67, or pharmaceutically acceptable salt thereof, wherein R₈ is —H.
69. The compound as defined in aspect 67, or pharmaceutically acceptable salt thereof, wherein R₈ is —F.
70. The compound as defined in aspect 67, or pharmaceutically acceptable salt thereof, wherein R₈ is methoxy.
71. The compound as defined in any one of aspects 1-63, or pharmaceutically acceptable salt thereof, wherein R₆, and R₇ are methyl, and R₈ is H.
72. The compound as defined in any one of aspects 1-63, or pharmaceutically acceptable salt thereof, wherein R₆, and R₇ are methyl, and R₈ is F.
73. The compound as defined in any one of aspects 1-63, or pharmaceutically acceptable salt thereof, wherein R₆, and R₇ are methyl, and R₈ is methoxy.
74. The compound as defined in any one of aspects 1-33, or pharmaceutically acceptable salt thereof, wherein R₄ is selected from

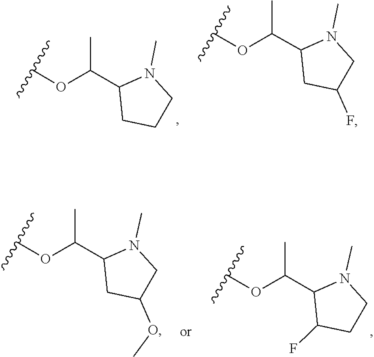

or a pharmaceutically acceptable salt thereof.
75. The compound according to aspect 74, wherein R₄ is selected from

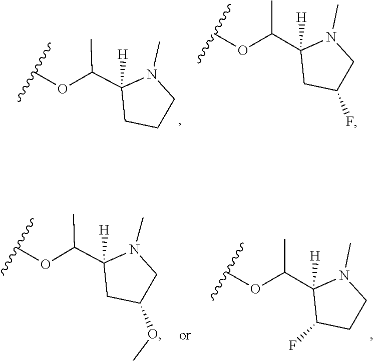

or a pharmaceutically acceptable salt thereof.
76. The compound according to aspect 74, wherein R₄ is selected from

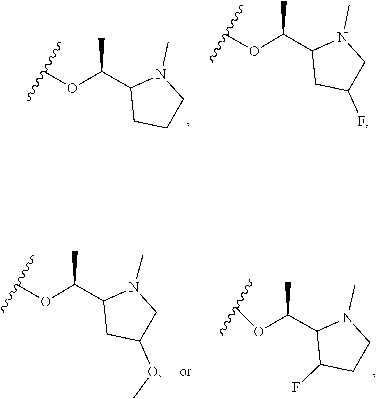

or a pharmaceutically acceptable salt thereof.
77. The compound according to aspect 74, wherein R₄ is selected from

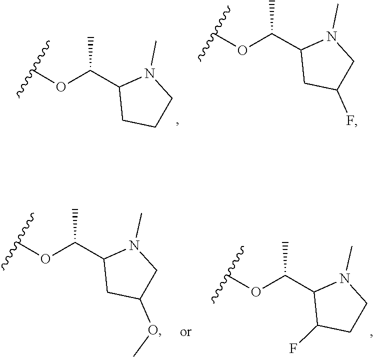

or a pharmaceutically acceptable salt thereof.
78. The compound according to aspect 75 or 76, wherein R₄ is selected from

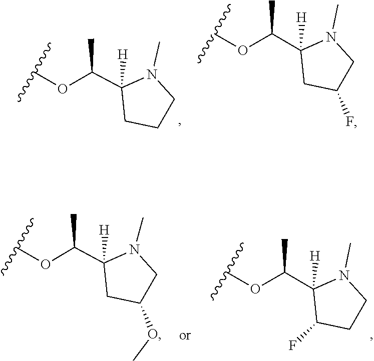

or a pharmaceutically acceptable salt thereof.
79. The compound according to aspect 1, selected from

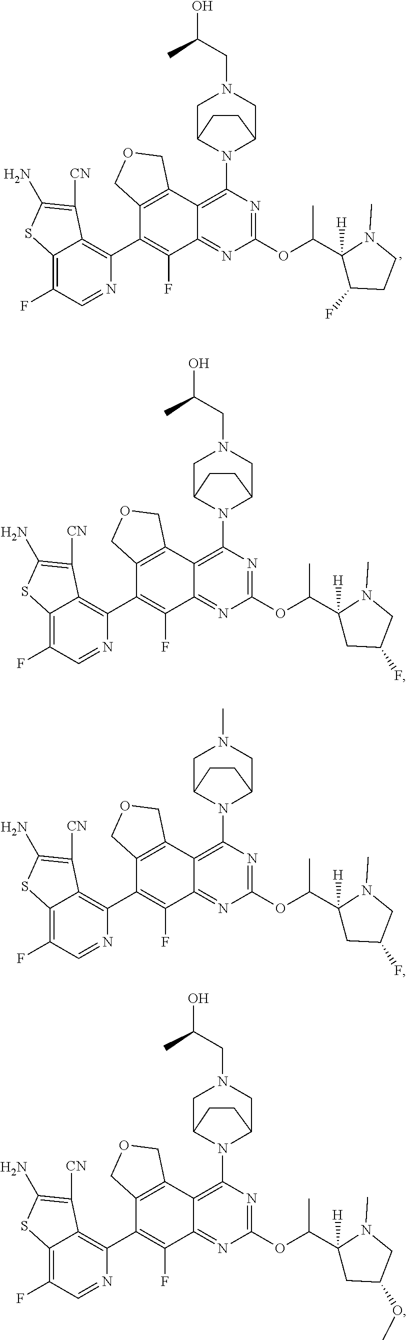 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1[C@@H](F)CCN1C ; CC(Oc1nc(N2C3CCC2CN(C[C@@H](C)O)C3)c2c3c(c(-c4ncc(F)c5sc(N)c(C#N)c45)c(F)c2n1)COC3)[C@@H]1C[C@@H](F)CN1C ; CC(Oc1nc(N2C3CCC2CN(C)C3)c2c3c(c(-c4ncc(F)c5sc(N)c(C#N)c45)c(F)c2n1)COC3)[C@@H]1C[C@@H](F)CN1C ; CO[C@@H]1C[C@@H](C(C)Oc2nc(N3C4CCC3CN(C[C@@H](C)O)C4)c3c4c(c(-c5ncc(F)c6sc(N)c(C#N)c56)c(F)c3n2)COC4)N(C)C1]

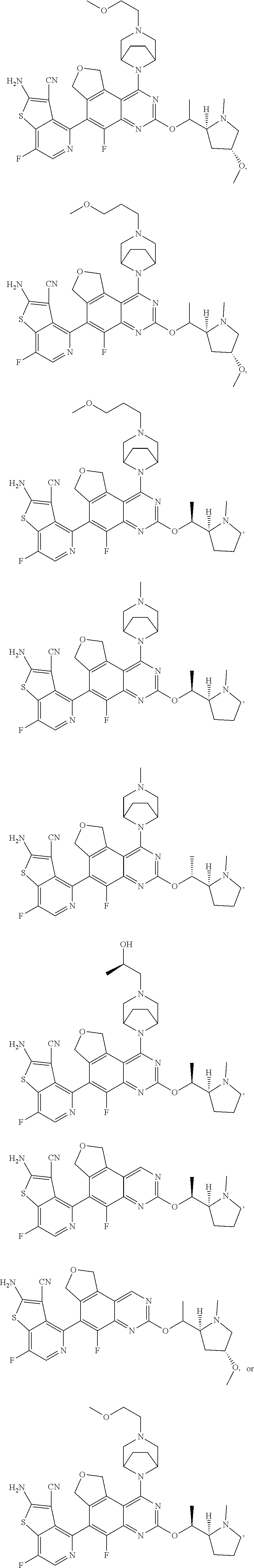

or a pharmaceutically acceptable salt thereof.
80. The compound according to aspect 1, selected from

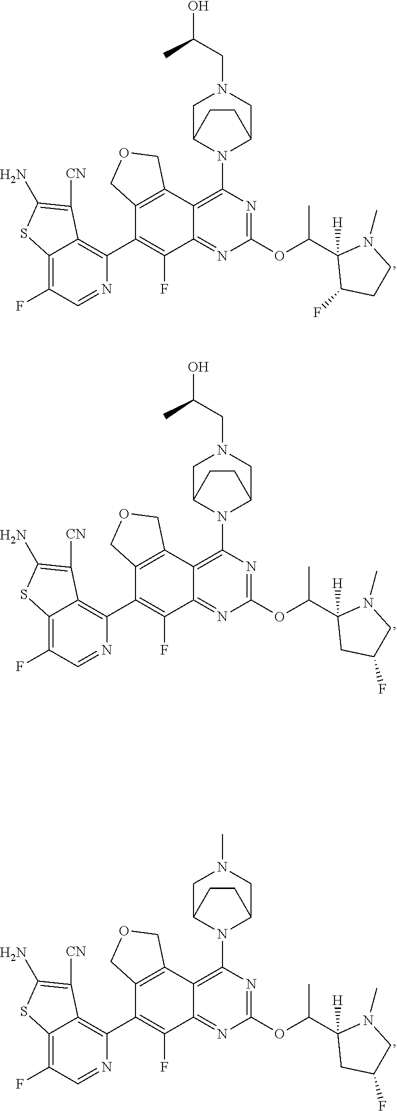 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1[C@@H](F)CCN1C ; CC(Oc1nc(N2C3CCC2CN(C[C@@H](C)O)C3)c2c3c(c(-c4ncc(F)c5sc(N)c(C#N)c45)c(F)c2n1)COC3)[C@@H]1C[C@@H](F)CN1C ; CC(Oc1nc(N2C3CCC2CN(C)C3)c2c3c(c(-c4ncc(F)c5sc(N)c(C#N)c45)c(F)c2n1)COC3)[C@@H]1C[C@@H](F)CN1C]

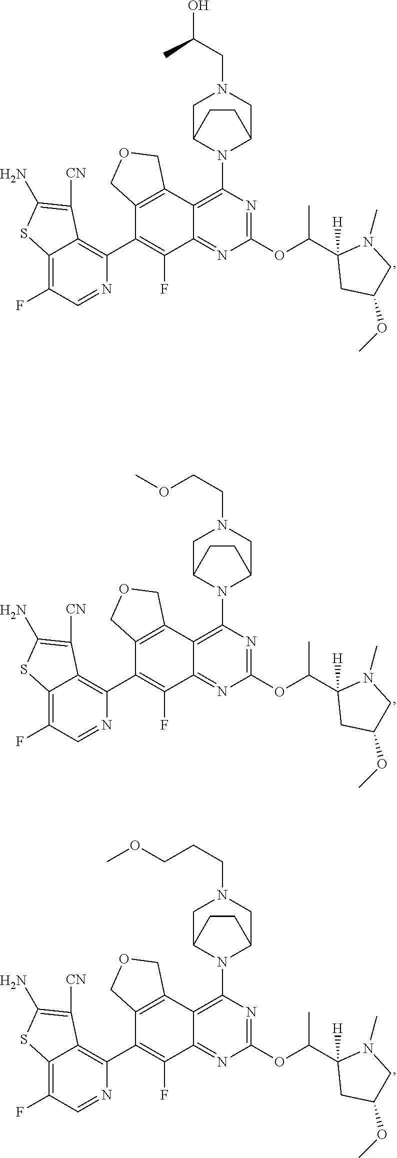 [SMILES: CO[C@@H]1C[C@@H](C(C)Oc2nc(N3C4CCC3CN(C[C@@H](C)O)C4)c3c4c(c(-c5ncc(F)c6sc(N)c(C#N)c56)c(F)c3n2)COC4)N(C)C1 ; COCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3 ; COCCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3]

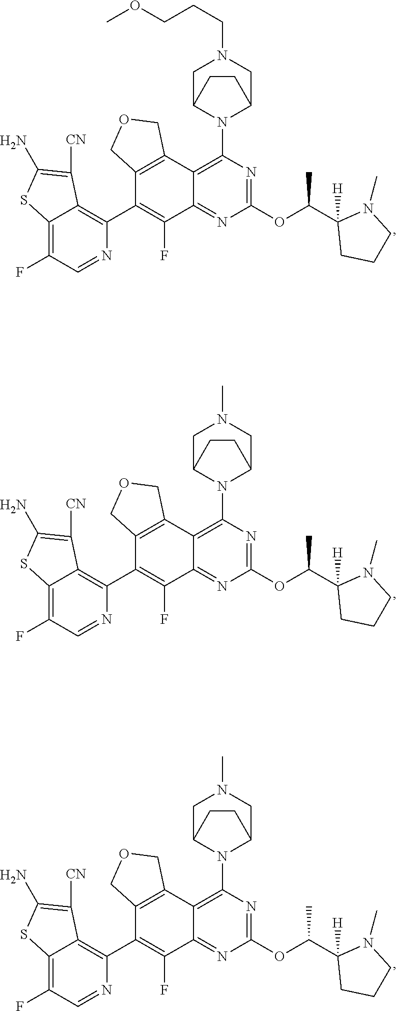 [SMILES: COCCCN1CC2CCC(C1)N2c1nc(O[C@@H](C)[C@@H]2CCCN2C)nc2c(F)c(-c3ncc(F)c4sc(N)c(C#N)c34)c3c(c12)COC3 ; C[C@H](Oc1nc(N2C3CCC2CN(C)C3)c2c3c(c(-c4ncc(F)c5sc(N)c(C#N)c45)c(F)c2n1)COC3)[C@@H]1CCCN1C ; C[C@@H](Oc1nc(N2C3CCC2CN(C)C3)c2c3c(c(-c4ncc(F)c5sc(N)c(C#N)c45)c(F)c2n1)COC3)[C@@H]1CCCN1C]

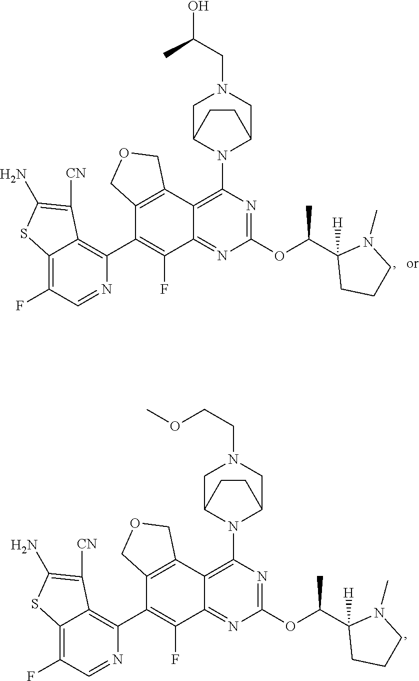 [SMILES: C[C@H](Oc1nc(N2C3CCC2CN(C[C@@H](C)O)C3)c2c3c(c(-c4ncc(F)c5sc(N)c(C#N)c45)c(F)c2n1)COC3)[C@@H]1CCCN1C ; COCCN1CC2CCC(C1)N2c1nc(O[C@@H](C)[C@@H]2CCCN2C)nc2c(F)c(-c3ncc(F)c4sc(N)c(C#N)c34)c3c(c12)COC3]

or a pharmaceutically acceptable salt thereof.
81. The compound according to aspect 1, selected from

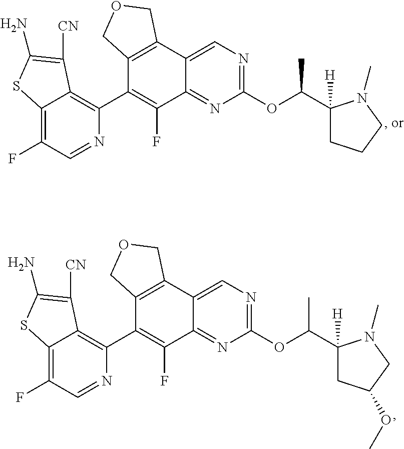 [SMILES: C[C@H](Oc1ncc2c3c(c(-c4ncc(F)c5sc(N)c(C#N)c45)c(F)c2n1)COC3)[C@@H]1CCCN1C ; CO[C@@H]1C[C@@H](C(C)Oc2ncc3c4c(c(-c5ncc(F)c6sc(N)c(C#N)c56)c(F)c3n2)COC4)N(C)C1]

or a pharmaceutically acceptable salt thereof.
82. The compound according to aspect 1, selected from

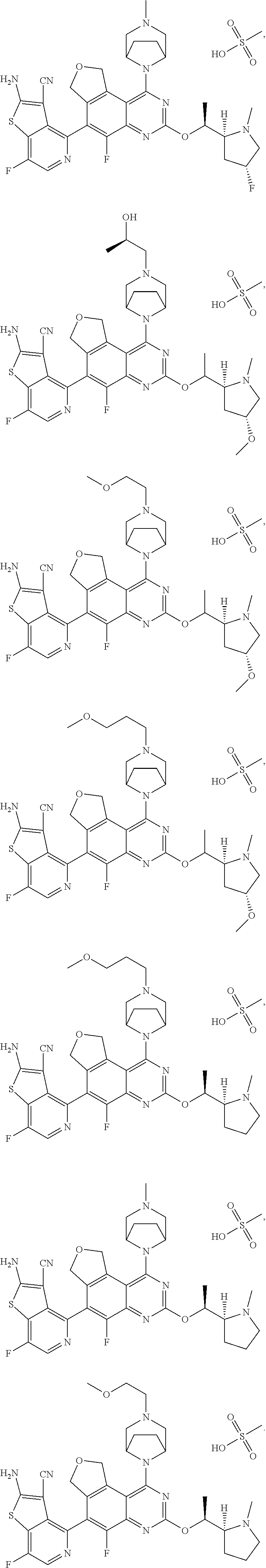

83. A pharmaceutical composition comprising a compound according to any one of aspects 1-82, or a pharmaceutically acceptable salt thereof, and a pharmaceutically acceptable carrier, diluent or excipient.
84. A method of treating a patient for cancer, comprising administering to a patient in need thereof, an effective amount of a pharmaceutical composition according to aspect 83, wherein the cancer is selected from lung cancer, pancreatic cancer, cervical cancer, esophageal cancer, endometrial cancer, ovarian cancer, cholangiocarcinoma, and colorectal cancer.
85. A method of treating a patient for cancer, comprising administering to a patient in need thereof, an effective amount of a compound according to any one of aspects 1-82, or a pharmaceutically acceptable salt thereof, wherein the cancer is selected from lung cancer, pancreatic cancer, cervical cancer, esophageal cancer, endometrial cancer, ovarian cancer, cholangiocarcinoma, and colorectal cancer.
86. The method according to aspects 84, or 85 wherein the patient has a cancer that was determined to have one or more cells expressing the KRas G12D mutant protein prior to administration of the compound or a pharmaceutically acceptable salt thereof.
87. The method according to any one of aspects 84-86, wherein the cancer is non-small cell lung cancer.
88. The method according to any one of aspects 84-86, wherein the cancer is colorectal cancer.
89. The method according to any one of aspects 84-86, wherein the cancer is pancreatic cancer.
90. The method according to any one of aspects 84, 85, or 87-89, wherein one or more cells express KRas G12D mutant protein.
91. A method of treating a patient with a cancer that has a KRas G12D mutation comprising administering to a patient in need thereof an effective amount of a compound according to any one of aspects 1-82, or a pharmaceutically acceptable salt thereof.
92. The method according to aspect 91, wherein the cancer is selected from lung cancer, pancreatic cancer, cervical cancer, esophageal cancer, endometrial cancer, mutant ovarian cancer, cholangiocarcinoma, and colorectal cancer.
93. The method according to aspect 92, wherein the cancer is non-small cell lung cancer.
94. The method according to aspect 92, wherein the cancer is colorectal cancer.
95. The method according to aspect 92, wherein the cancer is pancreatic cancer.
96. The method according to any one of aspects 84-95, wherein the patient is also administered an effective amount of one or more of a PD-1 inhibitor, a PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof.

In the above embodiments of the compounds of Formula I, the chemical drawings are shown flat without chiral information. These compounds often have multiple chiral centers and are contemplated to exist in various forms with various combinations of chiral centers. Additionally, these compounds have various enantiomers, diastereomers, and atropisomers that can exist and are included herein.

In an embodiment of a compound of Formula I or a pharmaceutically acceptable salt thereof, the compound is an isotopic derivative of any one of the compounds described herein or a pharmaceutically acceptable salt thereof.

It is understood that the isotopic derivative can be prepared using any of a variety of art-recognized techniques. For example, the isotopic derivatives can generally be prepared by carrying out the procedures disclosed in the schemes and/or in the examples described herein or a pharmaceutically acceptable salt thereof, by substituting an isotopically containing reagent for a non-isotopically containing reagent.

In an embodiment of a compound of Formula I or a pharmaceutically acceptable salt thereof, the compound is a deuterium containing compound of any one of the compounds described herein and pharmaceutically acceptable salts thereof.

In the compounds of this invention any atom not specifically designated as a particular isotope is meant to represent any stable isotope of that atom. Unless otherwise stated, when an atom is designated specifically as “H” or “hydrogen”, the atom is understood to have hydrogen at its natural abundance isotopic composition. Also, unless otherwise stated, when an atom is designated specifically as “D” or “deuterium”, the atom is understood to have deuterium at an abundance substantially greater than the natural abundance of deuterium, which is 0.015%.

A compound of Formula I or a pharmaceutically acceptable salt thereof, the compound is selected from

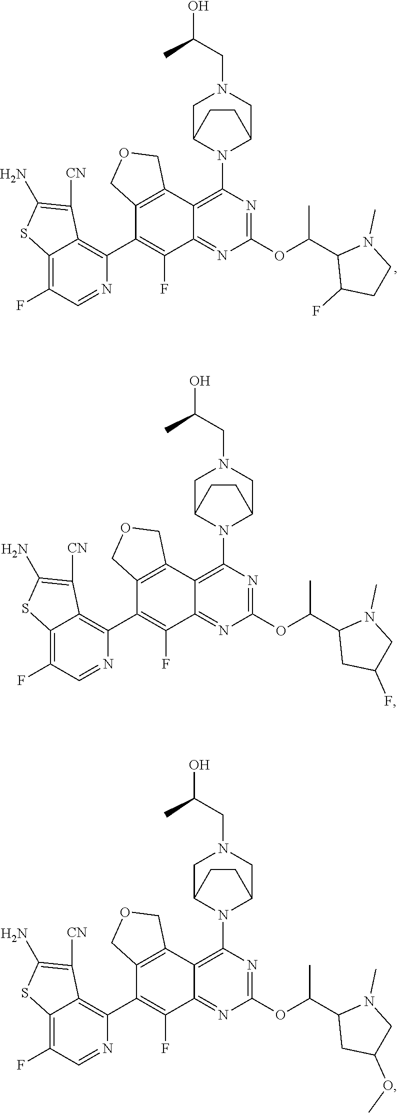 [SMILES: CC(Oc1nc(N2C3CCC2CN(C[C@@H](C)O)C3)c2c3c(c(-c4ncc(F)c5sc(N)c(C#N)c45)c(F)c2n1)COC3)C1C(F)CCN1C ; CC(Oc1nc(N2C3CCC2CN(C[C@@H](C)O)C3)c2c3c(c(-c4ncc(F)c5sc(N)c(C#N)c45)c(F)c2n1)COC3)C1CC(F)CN1C ; COC1CC(C(C)Oc2nc(N3C4CCC3CN(C[C@@H](C)O)C4)c3c4c(c(-c5ncc(F)c6sc(N)c(C#N)c56)c(F)c3n2)COC4)N(C)C1]

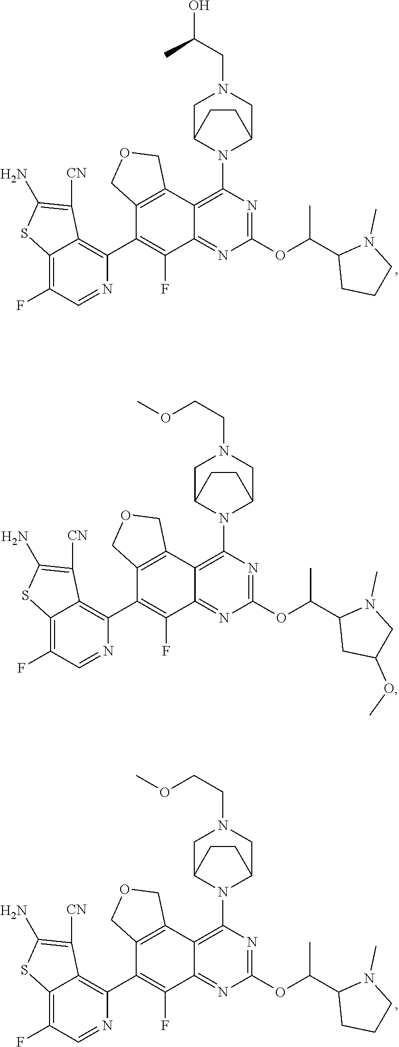 [SMILES: CC(Oc1nc(N2C3CCC2CN(C[C@@H](C)O)C3)c2c3c(c(-c4ncc(F)c5sc(N)c(C#N)c45)c(F)c2n1)COC3)C1CCCN1C ; COCCN1CC2CCC(C1)N2c1nc(OC(C)C2CC(OC)CN2C)nc2c(F)c(-c3ncc(F)c4sc(N)c(C#N)c34)c3c(c12)COC3 ; COCCN1CC2CCC(C1)N2c1nc(OC(C)C2CCCN2C)nc2c(F)c(-c3ncc(F)c4sc(N)c(C#N)c34)c3c(c12)COC3]

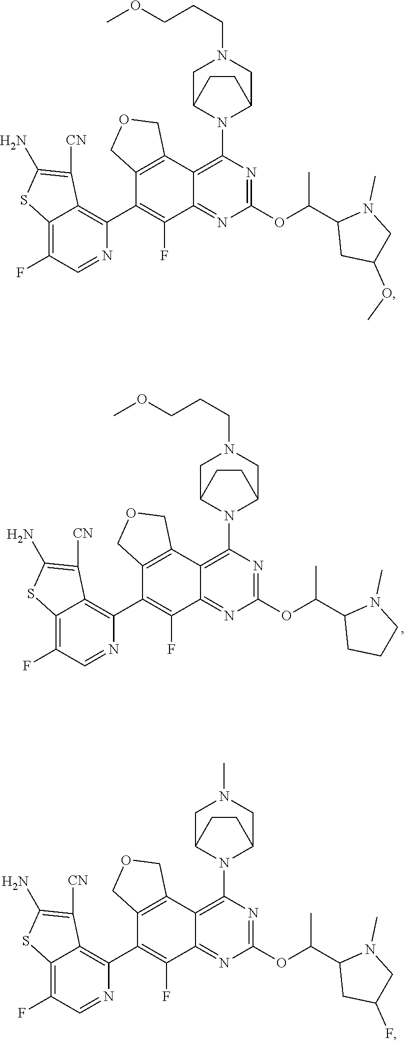 [SMILES: COCCCN1CC2CCC(C1)N2c1nc(OC(C)C2CC(OC)CN2C)nc2c(F)c(-c3ncc(F)c4sc(N)c(C#N)c34)c3c(c12)COC3 ; COCCCN1CC2CCC(C1)N2c1nc(OC(C)C2CCCN2C)nc2c(F)c(-c3ncc(F)c4sc(N)c(C#N)c34)c3c(c12)COC3 ; CC(Oc1nc(N2C3CCC2CN(C)C3)c2c3c(c(-c4ncc(F)c5sc(N)c(C#N)c45)c(F)c2n1)COC3)C1CC(F)CN1C]

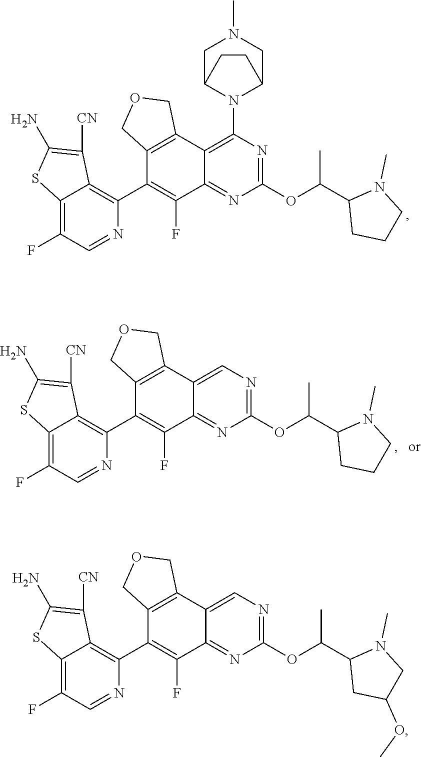 [SMILES: CC(Oc1nc(N2C3CCC2CN(C)C3)c2c3c(c(-c4ncc(F)c5sc(N)c(C#N)c45)c(F)c2n1)COC3)C1CCCN1C ; CC(Oc1ncc2c3c(c(-c4ncc(F)c5sc(N)c(C#N)c45)c(F)c2n1)COC3)C1CCCN1C ; COC1CC(C(C)Oc2ncc3c4c(c(-c5ncc(F)c6sc(N)c(C#N)c56)c(F)c3n2)COC4)N(C)C1]

or a pharmaceutically acceptable salt thereof.

The chemical drawings in the compounds above contain indications of chiral aspects of the specific compounds shown. However, the chemical drawings in the compounds above do not contain all the possible chiral features of these compounds and the chiral indications shown are not intended to exclude changes to the chiral aspects shown. Thus, alternate chiral versions of the compounds as well as different combinations of chiral attributes are contemplated and included herein.

Also provided herein are pharmaceutical compositions comprising a compound according to Formula I, or a pharmaceutically acceptable salt thereof, examples of which include, but are not limited to, the compounds disclosed herein, and a pharmaceutically acceptable carrier, diluent, or excipient.

Further provided herein are methods of treating cancer, comprising administering to a patient in need thereof, an effective amount of a compound according to Formula I, or a pharmaceutically acceptable salt thereof. In this method, the cancer can be lung cancer, colorectal cancer, pancreatic cancer, bladder cancer, cervical cancer, endometrial cancer, ovarian cancer, cholangiocarcinoma, gastric, or esophageal cancer. In this method, the cancer can more specifically be non-small cell lung cancer, pancreatic cancer, or colorectal cancer. In an embodiment the cancer can be non-small cell lung cancer. In an embodiment the cancer can be pancreatic cancer. In an embodiment the cancer can be colorectal cancer.

Also provided herein is a method of treating cancer, comprising administering to a patient in need thereof, an effective amount of a compound according to Formula I, or a pharmaceutically acceptable salt thereof, in which the cancer has one or more cells that express a mutant KRas G12D protein. In this method, the cancer can be non-small cell lung cancer, pancreatic cancer, or colorectal cancer, in which the cancer has one or more cells that express a KRas G12D mutant protein. In an embodiment, the cancer is non-small cell lung carcinoma in which the cancer has one or more cells that express a KRas G12D mutant protein. In an embodiment, the cancer is mutant pancreatic cancer in which the cancer has one or more cells that express a KRas G12D mutant protein. In an embodiment, the cancer is colorectal carcinoma in which the cancer has one or more cells that express a KRas G12D mutant protein. This method also includes treating KRas G12D mutant bearing cancers of other origins.

Further provided herein is a method of treating a patient with a cancer that has a KRas G12D mutation comprising administering to a patient in need thereof an effective amount of a compound according to Formula I or a pharmaceutically acceptable salt thereof. In this method, the cancer that has a KRas G12D mutation can be KRas G12D mutant lung cancer, KRas G12D mutant pancreatic cancer, KRas G12D mutant cervical cancer, KRas G12D mutant esophageal cancer, KRas G12D mutant endometrial cancer, KRas G12D mutant ovarian cancer, KRas G12D mutant cholangiocarcinoma, and KRas G12D mutant colorectal cancer. In an embodiment the cancer that has a KRas G12D mutation can be KRas G12D mutant non-small cell lung cancer. In an embodiment the cancer that has a KRas G12D mutation can be KRas G12D mutant pancreatic cancer. In an embodiment the cancer that has a KRas G12D mutation can be KRas G12D mutant colorectal cancer.

Additionally, provided herein is a method of modulating a mutant KRas G12D enzyme in a patient in need thereof, by administering a compound according to Formula I, or a pharmaceutically acceptable salt thereof. In one embodiment this method comprises inhibiting a human mutant KRas G12D enzyme.

Also provided herein is a method of treating cancer in a patient in need thereof, wherein the patient has a cancer that was determined to express the KRas G12D mutant protein. The method comprises administering to a patient an effective amount of a compound according to Formula I, or a pharmaceutically acceptable salt thereof. Typically, one or more biopsies containing one or more cancer cells are obtained, and subjected to sequencing and/or polymerase chain reaction (PCR). Circulating cell-free DNA can also be used, e.g. in advanced cancers. Non-limiting examples of sequencing and PCR techniques used to determine the mutational status (e.g., G12D mutational status, in one or more cancer cells or in circulating cell-free DNA) include direct sequencing, next-generation sequencing, reverse transcription polymerase chain reaction (RT-PCR), multiplex PCR, and pyrosequencing and multi-analyte profiling.

Further provided herein is a compound or a pharmaceutically acceptable salt thereof according to Formula I for use in therapy. The compound or a pharmaceutically acceptable salt thereof, can be for use in treating cancer. For this use in treating cancer, the cancer can be lung cancer, colorectal cancer, pancreatic cancer, bladder cancer, cervical cancer, endometrial cancer, ovarian cancer, cholangiocarcinoma, or esophageal cancer. The cancer can more specifically be non-small cell lung cancer, pancreatic cancer, or colorectal cancer. In an embodiment, the cancer is non-small cell lung cancer. In an embodiment, the cancer is pancreatic cancer. In an embodiment, the cancer is colorectal cancer. The cancer can have one or more cancer cells that express the mutant KRas G12D protein such as KRas G12D mutant lung cancer, KRas G12D mutant pancreatic cancer, KRas G12D mutant cervical cancer, KRas G12D mutant esophageal cancer, KRas G12D mutant endometrial cancer, KRas G12D mutant ovarian cancer, KRas G12D mutant cholangiocarcinoma, and KRas G12D mutant colorectal cancer. In these uses, the cancer is selected from: KRas G12D mutant non-small cell lung cancer, KRas G12D mutant colorectal cancer, and KRas G12D mutant pancreatic cancer. Additionally, the cancer can be non-small cell lung cancer, and one or more cells express KRas G12D mutant protein. Further, the cancer can be colorectal cancer, and one or more cells express KRas G12D mutant protein. Additionally, the cancer can be pancreatic cancer, and one or more cells express KRas G12D mutant protein. The patient can have a cancer that was determined to have one or more cells expressing the KRas G12D mutant protein prior to administration of the compound or a pharmaceutically acceptable salt thereof. The patient may have been treated with a different course of treatment prior to being treated as described herein.

The compounds provided herein according to Formula I, or a pharmaceutically acceptable salt thereof, may also be used in the manufacture of a medicament for treating cancer. When used in the manufacture of a medicament, the cancer can be lung cancer, colorectal cancer, pancreatic cancer, bladder cancer, cervical cancer, endometrial cancer, ovarian cancer, cholangiocarcinoma, or esophageal cancer. The cancer can more specifically be non-small cell lung cancer, pancreatic cancer, or colorectal cancer. In an embodiment, the cancer is non-small cell lung cancer. In an embodiment, the cancer is pancreatic cancer. In an embodiment, the cancer is colorectal cancer. The cancer can have one or more cancer cells that express the mutant KRas G12D protein. When the cancer cells express KRas G12D protein, the cancer can be selected from KRas G12D mutant non-small cell lung cancer, KRas G12D mutant colorectal cancer, and KRas G12D mutant pancreatic cancer.

Also provided herein is a method of treating cancer, comprising administering to a patient in need thereof, an effective amount of a compound according to Formula I, or a pharmaceutically acceptable salt thereof, and one or more of a PD-1 inhibitor, a PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, in which the cancer has one or more cells that express a mutant KRas G12D protein. Further provided herein is a compound according to Formula I, or a pharmaceutically acceptable salt thereof, for use in simultaneous, separate, or sequential combination with one or more of a PD-1 or PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, in the treatment of cancer. Additionally provided is a combination comprising a compound according to Formula I, or a pharmaceutically acceptable salt thereof, and one or more of a PD-1 or PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof, for simultaneous, separate, or sequential use in the treatment of cancer.

Also provided is a method of treating cancer, comprising administering to a patient in need thereof, an effective amount of a compound according to Formula I, or a pharmaceutically acceptable salt thereof, and a PD-1 or PD-L1 inhibitor, in which the cancer has one or more cells that express a mutant KRas G12D protein. Further provided is a compound according to Formula I, or a pharmaceutically acceptable salt thereof, for use in simultaneous, separate, or sequential combination with a PD-1 or PD-L1 inhibitor, for use in the treatment of cancer. Additionally provided is a combination comprising a compound according to Formula I, or a pharmaceutically acceptable salt thereof, and a PD-1 or PD-L1 inhibitor, for simultaneous, separate, or sequential use in the treatment of cancer. As used herein, the PD-1 or PD-L1 inhibitor can be pembrolizumab; the PD-1 or PD-L1 inhibitor can be nivolumab; the PD-1 or PD-L1 inhibitor can be cemiplimab; the PD-1 or PD-L1 inhibitor can be sintilimab; the PD-1 or PD-L1 inhibitor can be atezolizumab; the PD-1 or PD-L1 inhibitor can be avelumab; the PD-1 or PD-L1 inhibitor can be durvalumab; or the PD-1 or PD-L1 inhibitor can be lodapilimab. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; or the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and a CDK4/CDK6 inhibitor, or a pharmaceutically acceptable salt thereof, in which the cancer has one or more cells that express a mutant KRas G12D protein. Further provided is a compound according to Formula I, or a pharmaceutically acceptable salt thereof, for use in simultaneous, separate, or sequential combination with a CDK4/CDK6 inhibitor, or a pharmaceutically acceptable salt thereof, for use in the treatment of cancer, in which the cancer has one or more cells that express a mutant KRas G12D protein. Additionally provided is a combination comprising a compound according to Formula I, or a pharmaceutically acceptable salt thereof, and a CDK4/CDK6 inhibitor, or a pharmaceutically acceptable salt thereof, for simultaneous, separate, or sequential use in the treatment of cancer, in which the cancer has one or more cells that express a mutant KRas G12D protein. As used herein, the CDK4/CDK6 inhibitor can be abemaciclib; the CDK4/CDK6 inhibitor can be palbociclib; or the CDK4/CDK6 inhibitor can be ribociclib. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and an EGFR inhibitor, or a pharmaceutically acceptable salt thereof, in which the cancer has one or more cells that express a mutant KRas G12D protein. Further provided is a compound according to Formula I, or a pharmaceutically acceptable salt thereof, for use in simultaneous, separate, or sequential combination with an EGFR inhibitor, or a pharmaceutically acceptable salt thereof, for the treatment of cancer. Additional provided is a combination comprising a compound according to Formula I, or a pharmaceutically acceptable salt thereof, and an EGFR inhibitor, or a pharmaceutically acceptable salt thereof, for simultaneous, separate, or sequential use in the treatment of cancer. As used herein, the EGFR inhibitor can be erlotinib; the EGFR inhibitor can be afatinib; the EGFR inhibitor can be gefitinib; the EGFR inhibitor can be cetuximab. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; or the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and an ERK inhibitor, or a pharmaceutically acceptable salt thereof, in which the cancer has one or more cells that express a mutant KRas G12D protein. Also provided is a method of treating cancer, comprising administering to a patient in need thereof, an effective amount of a compound according to Formula I, or a pharmaceutically acceptable salt thereof, and an Aurora A inhibitor, in which the cancer has one or more cells that express a mutant KRas G12D protein. Further provided is a compound according to Formula I, or a pharmaceutically acceptable salt thereof, for use in simultaneous, separate, or sequential combination with an Aurora A inhibitor, or a pharmaceutically acceptable salt thereof, for the treatment of cancer, in which the cancer has one or more cells that express a mutant KRas G12D protein. Further provided is a compound according to Formula I, or a pharmaceutically acceptable salt thereof, for use in simultaneous, separate, or sequential combination with an ERK inhibitor, or a pharmaceutically acceptable salt thereof, for the treatment of cancer, in which the cancer has one or more cells that express a mutant KRas G12D protein. Additionally provided is a combination comprising a compound according to Formula I, or a pharmaceutically acceptable salt thereof, and an ERK inhibitor, or a pharmaceutically acceptable salt thereof, for simultaneous, separate, or sequential use in the treatment of cancer. As used herein, the ERK inhibitor can be LY3214996; the ERK inhibitor can be LTT462; or the ERK inhibitor can be KO-947. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and an Aurora A inhibitor, in which the cancer has one or more cells that express a mutant KRas G12D protein. Further provided is a compound according to Formula I, or a pharmaceutically acceptable salt thereof, for use in simultaneous, separate, or sequential combination with an Aurora A inhibitor, or a pharmaceutically acceptable salt thereof, for the treatment of cancer, in which the cancer has one or more cells that express a mutant KRas G12D protein. Additionally provided is a combination comprising a compound according to Formula I, or a pharmaceutically acceptable salt thereof, and an Aurora A inhibitor, for simultaneous, separate, or sequential use in the treatment of cancer. Additionally provided is a combination comprising a compound according to Formula I, or a pharmaceutically acceptable salt thereof, and an Aurora A inhibitor, for simultaneous, separate, or sequential use in the treatment of cancer. As used herein, the Aurora A inhibitor can be alisertib, tozasertib, (2R, 4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, (2R, 4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid: 2-methylpropan-2-amine (1:1) salt, and (2R, 4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt, or a pharmaceutically acceptable salt thereof. In one embodiment, the Aurora A inhibitor is (2R, 4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and a SHP2 inhibitor, in which the cancer has one or more cells that express a mutant KRas G12D protein. Further provided is a compound according to Formula I, or a pharmaceutically acceptable salt thereof, for use in simultaneous, separate, or sequential combination with a SHP2 inhibitor, or a pharmaceutically acceptable salt thereof, for the treatment of cancer, in which the cancer has one or more cells that express a mutant KRas G12D protein. Additionally provided is a combination comprising a compound according to Formula I, or a pharmaceutically acceptable salt thereof, and a SHP2 inhibitor, for simultaneous, separate, or sequential use in the treatment of cancer. As used herein, the SHP2 inhibitor, or a pharmaceutically acceptable salt thereof, can be a Type I SHP2 Inhibitor or a Type II SHP2 Inhibitor. Examples of Type I SHP2 inhibitors include, but are not limited to, PHPS1, GS-493, NSC-87877, NSC-117199, and Cefsulodin, and pharmaceutically acceptable salts thereof. Examples of Type II SHP2 inhibitors include, but are not limited to, JAB-3068, JAB-3312, RMC-4550, RMC-4630, SHP099, SHP244, SHP389, SHP394, TNO155, RG-6433, and RLY-1971, and pharmaceutically acceptable salts thereof. Additional examples of SHP2 inhibitors include, but are not limited to, BBP-398, IACS-15509, IACS-13909, X37, ERAS-601, SH3809, HBI-2376, ETS-001, and PCC0208023, and pharmaceutically acceptable salts thereof. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins.

Also provided is a method of treating cancer, comprising administering to a patient in need thereof, an effective amount of a compound according to Formula I, or a pharmaceutically acceptable salt thereof, and a platinum agent, in which the cancer has one or more cells that express a mutant KRas G12D protein. Further provided is a compound according to Formula I, or a pharmaceutically acceptable salt thereof, for use in simultaneous, separate, or sequential combination with a platinum agent, or a pharmaceutically acceptable salt thereof, for the treatment of cancer, in which the cancer has one or more cells that express a mutant KRas G12D protein. Additionally provided is a combination comprising a compound according to Formula I, or a pharmaceutically acceptable salt thereof, and a platinum agent, for simultaneous, separate, or sequential use in the treatment of cancer. As used herein, the platinum agent can be cisplatin; the platinum agent can be carboplatin; or the platinum agent can be oxaliplatin. As described herein, the cancer can be non-small cell lung carcinoma, in which the cancer has one or more cells that express a KRas G12D mutant protein; the cancer can be colorectal carcinoma in which the cancer has one or more cells that express a KRas G12D mutant protein; the cancer can be mutant pancreatic cancer in which the cancer has one or more cells that express a KRas G12D mutant protein. This method also includes treating KRas G12D mutant bearing cancers of other origins. As used herein, the platinum agent can be cisplatin; the platinum agent can be carboplatin; or the platinum agent can be oxaliplatin.

Also provided is a method of treating cancer, comprising administering to a patient in need thereof, an effective amount of a compound according to Formula I, or a pharmaceutically acceptable salt thereof, and pemetrexed, in which the cancer has one or more cells that express a mutant KRas G12D protein. Further provided is a compound according to Formula I, or a pharmaceutically acceptable salt thereof, for use in simultaneous, separate, or sequential combination with pemetrexed, for the treatment of cancer, in which the cancer has one or more cells that express a mutant KRas G12D protein. Additionally provided is a combination comprising a compound according to Formula I, or a pharmaceutically acceptable salt thereof, and pemetrexed, for simultaneous, separate, or sequential use in the treatment of cancer, in which the cancer has one or more cells that express a mutant KRas G12D protein. As described herein, the cancer has one or more cells that express a KRas G12D mutant protein. Further, a platinum agent can also be administered to the patient (and the platinum agent can be cisplatin, carboplatin, or oxaliplatin). As described herein, the cancer can be colorectal carcinoma in which the cancer has one or more cells that express a KRas G12D mutant protein or the cancer can be mutant pancreatic cancer in which the cancer has one or more cells that express a KRas G12D mutant protein. This method also includes treating KRas G12D mutant bearing cancers of other origins.

The term “pharmaceutically acceptable salt” as used herein refers to a salt of a compound considered to be acceptable for clinical and/or veterinary use. Examples of pharmaceutically acceptable salts and common methodology for preparing them can be found in “Handbook of Pharmaceutical Salts: Properties, Selection and Use” P. Stahl, et al., 2nd Revised Edition, Wiley-VCH, 2011 and S. M. Berge, et al., “Pharmaceutical Salts”, Journal of Pharmaceutical Sciences, 1977, 66(1), 1-19.

Pharmaceutical compositions containing the compounds of Formula I as described herein may be prepared using pharmaceutically acceptable additives. The term “pharmaceutically acceptable additive(s)” as used herein for the pharmaceutical compositions, refers to one or more carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

As used herein, the term “effective amount” refers to an amount that is a dosage, which is effective in achieve a desired therapeutic result such as treating a disorder or disease, like a cancerous lesion or progression of abnormal cell growth and/or cell division. Factors considered in the determination of an effective amount or dose of a compound include: whether the compound or its salt will be administered; the co-administration of other agents, if used; the species of patient to be treated; the patient's size, age, gender, and general health; the degree of involvement or stage and/or the severity of the disorder; the response of the individual patient; the mode of administration; the bioavailability characteristics of the preparation administered; the dose regimen selected; and the use of other concomitant medication.

A treating physician, veterinarian, or other medical person will be able to determine an effective amount of the compound for treatment of a patient in need. Pharmaceutical compositions can be formulated as a tablet or capsule for oral administration, a solution for oral administration, or an injectable solution. The tablet, capsule, or solution can include a compound of the present invention in an amount effective for treating a patient in need of treatment for cancer.

As used herein, the terms “treating”, “to treat”, or “treatment”, includes slowing, controlling, delaying, reducing, stopping, reversing, preventing, or ameliorating the progression or severity of an existing symptom, disorder, condition, which can include specifically slowing the growth of a cancerous lesion or progression of abnormal cell growth and/or cell division. Treating does not necessarily indicate a total elimination of all disorder or disease symptoms.

As used herein, the term “patient” refers to a mammal in need of treatment. Specifically, the patient can be a human that is in need of treatment for cancer, for example, KRas G12D mutant bearing cancers.

Certain abbreviations are defined as follows: “ACN” refers to acetonitrile; “AcOH” or “HOAc” refer to acetic acid; AIBN” refers to azobisisobutyronitrile; “Alloc” refers to the allyloxycarbonyl group; “aq.” refers to aqueous; “atm” refers to atmosphere or atmospheres; “Boc-Gly-OH” refers to N-(tert-butoxycarbonyl)glycine; “BrettPhos” refers to 2-dicyclohexylphosphino-3, 6-dimethoxy-2′, 4′, 6′-triisopropyl-1, 1′-biphenyl; “BroP” refers to bromo tris(dimethylamino) phosphonium hexafluorophosphate; “Cbz” refers to the benzyloxycarbonyl group; “Cbz-Cl” refers to benzyl chloroformate; “conc.” refers to concentrated; “CSI” refers to chlorosulfonyl isocyanate; “CV” refers to column volumes; “DCM” refers to dichloromethane; “DIAD” refers to diisopropyl azodicarboxylate; “DIBAL-H” refers to diisobutylaluminum hydride; “DIEA” and “DIPEA” refer to N, N-diisopropyl ethylamine; “(dippf)Rh(cod)BF₄” refers to [1, 4-bis(diphenylphosphino)butane](1, 5-cyclooctadiene)rhodium(I) tetrafluoroborate; “DMAP” refers to 4-dimethylaminopyridine; “DMEA” refers to N, N-dimethylethylamine; “DMEM” refers to Dulbecco's modified Eagle's medium; “DMF” refers to N, N-dimethylformamide; “DMSO” refers to dimethylsulfoxide; “DNA” refers to deoxyribonucleic acid; “DPEPhosPdCl₂” refers to dichlorobis(diphenylphosphinophenyl) ether palladium (II); “DTT” refers to dithiothreitol; “EDTA” refers to ethylenediaminetetraacetic acid; “EGTA” refers to ethylene glycol-bis(b-aminoethyl ether)-N, N, N′, N′-tetraacetic acid; “ELISA” refers to enzyme-linked immunosorbent assay; “ERK” refers to extracellular signal-regulated kinases; “EtOAc” refers to ethyl acetate; “Et₂O” refers to diethyl ether; “EtOH” refers to ethanol; “FA” refers to formic acid; “FBS” refers to fetal bovine serum; “Fmoc” refers to the fluorenylmethyloxycarbonyl group; “GDP” refers to guanosine diphosphate; “GTP” refers to guanosine triphosphate; “h” refers to hour or hours; “HATU” refers to 1-[Bis(dimethylamino)methylene]-1H-1, 2, 3-triazolo[4, 5-b]pyridinium 3-oxide hexafluorophosphate; “Hex” or “hex” refers to hexane or hexanes; “HPLC” refers to high-performance liquid chromatography; “HRP” refers to horseradish peroxidase; “IPA” refers to isopropyl alcohol; “IPAm” refers to isopropyl amine; “KOAc” refers to potassium acetate; “LC-ES/MS” refers to liquid chromatograph-electrospray mass spectrometry; “LC-MS” refers to liquid chromatography mass spectrometry; “LiHMDS” refers to lithium bis(trimethylsilyl)amide; “L-prolinol” refers to [(2S)-pyrrolidin-2yl]methanol; “MAPK” refers to mitogen-activated protein kinases; “mCPBA” refers to 3-chloro-peroxybenzoic acid; “Me” refers to a methyl group; “MeOH” refers to methanol; “min” refers to minute or minutes; “MTBE” refers to methyl tert-butyl ether; “NaBH(OAc)₃ refers to sodium triacetoxyborohydride; “NaOMe” refers to sodium methoxide; “NBS” refers to N-bromosuccinimide; “NCS” refers to N-chlorosuccinimide; “N-methyl-L-prolinol” refers to [(2S)-1-methylpyrrolidin-2-yl]methanol; “NMM” refers to N-methylmorpholine; “NMP” refers to 1-methylpyrrolidin-2-one; “NIS” refers to N-iodosuccinimide; “PCR” refers to polymerase chain reaction; “Pd-117” refers to dichloro[bis(2-(diphenylphosphino)phenyl)ether]palladium(II), CAS 205319-06-8; “Pd-118” refers to 1, 1′-bis(di-tert-butylphosphino)ferrocene palladium dichloride, CAS 95408-45-0; “Pd₂(dba)₃” refers to tris(dibenzylideneacetone)dipalladium(0); “Pd(dppf)Cl₂” refers to [1, 1′-bis(diphenylphosphino)ferrocene]dichloropalladium(II); “Pd(OAc)₂ refers to palladium (II) acetate; Pd(PPh₃)₄ refers to tetrakis(triphenylphosphine)palladium(0); “PE” refers to petroleum ether or diethyl ether; “Ph” refers to phenyl; “RBF” refers to round bottom flask; “RPMI” refers to Roswell Park Memorial Institute; “RT” refers to room temperature; “RuPhos” refers to 2-dicyclohexylphosphino-2′, 6′-diisopropoxy-1, 1′-biphenyl, CAS 787618-22-8; “sat.” refers to saturated; “SCX” refers to strong cation exchange; “Selectfluor™ refers to 1-chloromethyl-4-fluoro-1, 4-diazoniabicyclo[2.2.2]octane bis(tetrafluoroborate), “SPE” refers to solid phase extraction; “SPhos” refers to 2-dicyclohexylphosphino-2′, 6′-dimethoxy-1, 1′-biphenyl; “TBAF” refers to tetrabutylammonium fluoride; “TBDMSCl” refers to tert-butyldimethylsilyl chloride; “TBDMS” refers to the tert-butyldimethylsilyl group; “tBu” refers to the tert-butyl group; “t-BuOH” refers to tert-butanol or tert-butyl alcohol; A” refers to triethylamine; “TES” refers to triethylsilane; “Tf₂O” refers to trifluoromethanesulfonic anhydride; “TFA” refers to trifluoracetic acid; “THF” refers to tetrahydrofuran; “TMEDA” refers to tetramethylethylenediamine; “t_{R}” refers to retention time; “XantPhos” refers to 4, 5-bis(diphenylphosphino)-9, 9-dimethylxanthene; “XPhos” refers to 2-(dicyclohexylphosphino)-2′, 4′, 6′-tri-isopropyl-1, 1′-biphenyl; “XPhos Palladacycle G2” refers to chloro(2-dicyclohexylphosphino-2′, 4′, 6′-triisopropyl-1, 1′-biphenyl)[2-(2′-amino-1, 1′-biphenyl)]palladium(II), CAS 1310584-14-5; “XPhos Palladacycle Gen.4” or “XPhos Pd G4” refer to methanesulfonato(2-dicyclohexylphosphino-2′, 4′, 6′-triisopropyl-1, 1′-biphenyl)(2′-methylamino-1, 1′-biphenyl-2-yl)palladium(II), CAS 1599466-81-5.

Individual isomers, enantiomers, diastereomers, and atropisomers may be separated or resolved at any convenient point in the synthesis of compounds listed below, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994). The molecules described herein include compounds that are atropisomers and which can exist in different conformations or as different rotomers. Atropisomers are compounds that exist in different conformations arising from restricted rotation about a single bond. Atropisomers can be isolated as separate chemical species if the energy barrier to rotation about the single bond is sufficiently high that the rate of interconversion is slow enough to allow the individual rotomers to be separated from each other. This description is intended to include all of the isomers, enantiomers, diastereomers, and atropisomers possible for the compounds disclosed herein or that could be made using the compounds disclosed herein. In the molecules described herein, only molecules in which the absolute conformation of a chiral center (or atropisomer conformation) is known have used naming conventions or chemical formula that are drawn to indicate the chirality or atropisomerism. Those of skill in the art will readily understand when other chiral centers are present in the molecules described herein and be able to identify the same.

Compounds of any one of Formula I that are chemically capable of forming salts are readily converted to and may be isolated as a pharmaceutically acceptable salt. Salt formation can occur upon the addition of a pharmaceutically acceptable acid to form the acid addition salt. Salts can also form simultaneously upon deprotection of a nitrogen or oxygen, i.e., removing the protecting group. Examples, reactions and conditions for salt formation can be found in Gould, P. L., “Salt selection for basic drugs,” International Journal of Pharmaceutics, 33: 201-217 (1986); Bastin, R. J., et al. “Salt Selection and Optimization Procedures for Pharmaceutical New Chemical Entities,” Organic Process Research and Development, 4: 427-435 (2000); and Berge, S. M., et al., “Pharmaceutical Salts,” Journal of Pharmaceutical Sciences, 66: 1-19, (1977).

The compounds of the present invention, or salts thereof, may be prepared by a variety of procedures, some of which are illustrated in the Schemes, Preparations, and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

### Preparation 1

### 5-Fluoroisobenzofuran-1(3H)-one

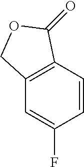 [SMILES: O=C1OCc2cc(F)ccc21]

To a stirred mixture of (2-bromo-5-fluorophenyl)methanol 500 g, 2.44 mol) and TEA (474.6 mL, 3.41 mol, 1.4 eq.) in ACN (2500 mL) was added Pd(OAc)₂ (10.95 g, 48.77 mmol, 0.02 eq.) and XantPhos (42.33 g, 73.16 mmol, 0.03 equiv.) at RT, then stirred for 3 days at 120° C. under 10 atm of carbon monoxide. The reaction was cooled to RT and concentrated. The residue was diluted with H₂O (1,000 mL), then extracted with EtOAc (2×2000 mL). The combined organic layers were washed with brine (2×1,000 mL), dried over anhydrous Na₂SO₄, filtered and concentrated. The residue was triturated with 10:1 hexanes/EtOAc (1,100 mL) and then filtered. The filter cake was dried at 50° C. for ˜18 h to obtain the title compound as a yellow solid (300 g, 81%). MS (ES) m/z=153 (M+1).

### Preparation 2

### 4-Bromo-5-fluoro-6-nitroisobenzofuran-1(3H)-one

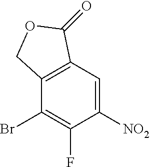 [SMILES: O=C1OCc2c1cc([N+](=O)[O-])c(F)c2Br]

To a stirred mixture of 5-fluoroisobenzofuran-1(3H)-one (300 g, 1.97 mol) in H₂SO₄ (1,500 mL) was added HNO₃ (273.38 g, 4.348 mol, 2.2 eq.) dropwise at 65° C. The reaction was stirred for 1 h then cooled to RT. 1,3-dibromo-5,5-dimethylimidazolidine-2,4-dione (2,255.43 g, 7.88 mol, 4 eq.) was added in portions over 20 min and was stirred at RT for ˜18 h. The mixture was poured onto ice/water (pre-treated with 3 kg Na₂SO₃) and filtered. The filter cake was dissolved in EtOAc (3,000 mL), washed with sat. aq. Na₂CO₃ (2×1,000 mL), brine (2×1,000 mL), dried over anhydrous Na₂SO₄ and concentrated. The residue was triturated with 10:1 hexanes/EtOAc (660 mL) and was filtered and dried at 50° C. for ˜18 h to obtain the title compound as a yellow solid (270 g, 49%) which was used in a subsequent step without further purification. ¹H NMR (400 MHz, DMSO-d₆) δ 8.58 (s, 1H), 5.51 (s, 2H).

### Preparation 3

### 4-Bromo-5-fluoro-6-nitro-1,3-dihydroisobenzofuran

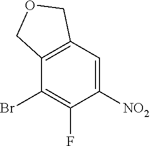 [SMILES: O=[N+]([O-])c1cc2c(c(Br)c1F)COC2]

To a stirred mixture of 4-bromo-5-fluoro-6-nitroisobenzofuran-1(3H)-one (270 g, 978 mmol) in DCM (2,500 mL) was added DIBAL-H (1M in THF, 1,467 mL, 1.467 mol, 1.5 eq.) dropwise at −78° C. under N2. The reaction was stirred for 5 h at −78° C., then was quenched with 5N NaOH (300 mL) at −78° C. The resulting mixture was allowed to warm to RT, then was concentrated. The residue was diluted with EtOAc (2,500 mL), washed with brine (2×1,000 mL) and dried over anhydrous Na₂SO₄ and concentrated. The residue was triturated with 10:1 hexanes/EtOAc (550 mL) and filtered. The solids were dried (190 g, 683.4 mmol) then dissolved in DCM (1,500 mL) and treated dropwise with Et₃SiH (662 mL, 4.10 mol, 6 eq.) at 0° C. The reaction was stirred for 20 min at 0° C. TFA (152 mL, 2.05 mol, 3 eq.) was added dropwise at 0° C. The ice bath was removed, and the reaction was stirred at RT for ˜18 h. The reaction was concentrated to an oil, which was diluted with EtOAc (2,000 mL), washed with sat. aq. Na₂CO₃ (2×500 mL) and brine (2×500 mL), dried over anhydrous Na₂SO₄, filtered and concentrated to obtain the title compound (110 g, 42%) which was used in a subsequent step without further purification. ¹H NMR (400 MHz, DMSO-d₆) δ 8.16 (d, J=6.2 Hz, 1H), 5.18-5.15 (m, 2H), 5.11-5.06 (m, 2H).

### Preparation 4

### 7-Bromo-6-fluoro-1,3-dihydroisobenzofuran-5-amine

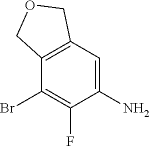 [SMILES: Nc1cc2c(c(Br)c1F)COC2]

To a stirred mixture of 4-bromo-5-fluoro-6-nitro-1,3-dihydroisobenzofuran (110 g, 420 mmol) and NH₄Cl (112.3 g, 2.10 mol, 5 eq.) in EtOH (1,000 mL) and H₂O (200 mL) was added Fe (117.22 g, 2.09 mol, 5 eq.) in portions at RT, then stirred for ˜18 h at 80° C. The mixture was filtered and concentrated. The mixture was diluted with H₂O (500 mL) and extracted with EtOAc (2×1,000 mL). The combined organic layers were washed with brine (2×500 mL), dried over anhydrous Na₂SO₄, filtered and concentrated. The residue was purified on silica (25% to 50% EtOAc/Hex) to afford the title compound (70 g, 72%) as a yellow solid. MS (ES) m/z=231 (M+1).

### Preparation 5

### Ethyl N-[(7-bromo-6-fluoro-1,3-dihydroisobenzofuran-5-yl)carbamothioyl]carbamate

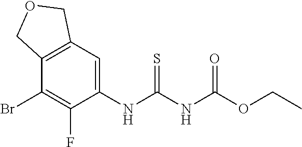 [SMILES: CCOC(=O)NC(=S)Nc1cc2c(c(Br)c1F)COC2]

A solution of 7-bromo-6-fluoro-1,3-dihydroisobenzofuran-5-amine (20.4 g, 87.9 mmol) in DCM (550 mL) was charged with ethoxycarbonyl isothiocyanate (9.7 mL, 82 mmol, 0.93 eq.) slowly via addition funnel and subsequently stirred at RT for ˜4 h. The solids were filtered. The filtrate was concentrated, suspended in DCM (100 mL) and hexanes (350 mL) and stirred at RT. The resultant filtered solids and previous filtered solids were dried under vacuum at 50° C. for 2 h. The batches were combined to obtain the title compound (32.6 g, quantitative) as a white solid. MS (ES) m/z=363 (M+1).

### Preparation 6

### Ethyl (((7-bromo-6-fluoro-1,3-dihydroisobenzofuran-5-yl)amino)(ethylthio)methylene)carbamate

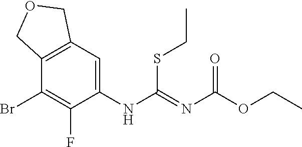 [SMILES: CCOC(=O)/N=C(/Nc1cc2c(c(Br)c1F)COC2)SCC]

A 2 L 3-necked RBF, equipped with an overhead stirrer, dropping funnel and thermocouple was charged with a suspension of ethyl N-[(7-bromo-6-fluoro-1,3-dihydroisobenzofuran-5-yl)carbamothioyl]carbamate (32.6 g, 89.8 mmol) and acetone (450 mL). To this was added solid K₂CO₃ (37.2 g, 269 mmol, 3.00 eq.) in several portions, followed by the dropwise addition of EtI (7.2 mL, 90 mmol, 1.0 eq.) over 20 min. The mixture was stirred at RT for ˜18 h. The solids were filtered and the filtrate was concentrated and partitioned between DCM (500 mL) and H₂O (500 mL). The organics were further washed with brine and dried over anhydrous Na₂SO₄, filtered and concentrated. The residue was purified on silica (0 to 30% EtOAc/Hex) to obtain the title compound (30.9 g, 85.6%) as a white solid. MS (ES) m/z=391 (M+1).

### Preparation 7

### 6-Bromo-3-(ethylthio)-5-fluoro-7,9-dihydrofuro[3,4-f]quinazolin-1-ol

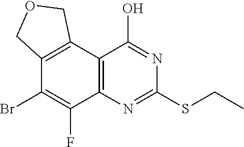 [SMILES: CCSc1nc(O)c2c3c(c(Br)c(F)c2n1)COC3]

A 2 L 4-necked RBF was equipped with an overhead stirrer, dropping funnel, N2 inlet and thermocouple and was purged with N2. NMP (anhydrous, 300 mL) was added. The mixture was heated to 175° C. In a second flask, ethyl (((7-bromo-6-fluoro-1,3-dihydroisobenzofuran-5-yl)amino)(ethylthio)methylene)carbamate (22.63 g, 57.83 mmol) and NMP (anhydrous, 100 mL) were combined and stirred under N2 until a homogeneous solution was obtained. When the first flask had reached 175° C., the contents of the second flask were poured into the dropping funnel and were added dropwise but rapidly to the hot NMP. After 30 min, the heat was turned off and the reaction cooled to 45° C. H₂O (500 mL) was slowly added and the mixture was stirred at RT for 1 h. The solids were filtered, rinsed with H₂O (300 mL) and dried under vacuum at 50° C. for ˜18 h to afford the title compound (15.2 g, 73%) as an off-white solid. MS (ES) m/z=363 (M+1).

### Preparation 8

### 6-Bromo-1-chloro-3-(ethylthio)-5-fluoro-7,9-dihydrofuro[3,4-f]quinazoline

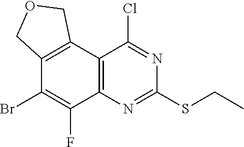 [SMILES: CCSc1nc(Cl)c2c3c(c(Br)c(F)c2n1)COC3]

A 5 L 3-necked RBF, equipped with a dropping funnel, thermocouple and an overhead stirrer was charged with a solution of DMF (50 mL, 646 mmol, 4 eq.) in DCM (1,000 mL) and was placed in an ice/water bath and cooled to −4° C. Oxalyl chloride (50.0 mL, 576 mmol, 4 eq.) was added dropwise via addition funnel over −40 min. When the addition was complete, the reaction was stirred at −4° C. for 15 min. Solid 6-bromo-3-(ethylthio)-5-fluoro-7,9-dihydrofuro[3,4-f]quinazolin-1-ol (50.4 g, 140 mmol) was added in several portions to the reaction mixture and the resulting suspension was stirred at −4° C. for 30 min. The ice bath was removed and the reaction was allowed to warm to RT and stir for 1 h. Then H₂O (1 L) was added and the mixture was stirred for 15 min. The mixture was partitioned and the organic layer was washed with brine (1 L) and dried over anhydrous Na₂SO₄, filtered and concentrated. The residue was purified on silica, eluting with DCM/Hex (60% to 90%) to obtain the title compound (45.1 g, 89%) as a white solid. MS (ES) m/z=363 (M+1).

### Preparation 9

### 6-Bromo-3-(ethylthio)-5-fluoro-7,9-dihydrofuro[3,4-f]quinazoline

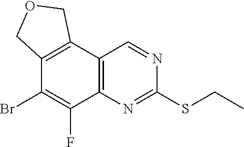 [SMILES: CCSc1ncc2c3c(c(Br)c(F)c2n1)COC3]

To a mixture of 6-bromo-1-chloro-3-(ethylthio)-5-fluoro-7,9-dihydrofuro[3,4-f]quinazoline (6.50 g, 17.9 mmol) and tetramethylethylenediamine (2.28 g, 19.7 mmol) in THE (100 mL) was added [1,1′-bis(diphenylphosphino)ferrocene]dichloropalladium(II) (0.73 g, 1.00 mmol) and sodium cyanoborohydride (in portions, 2.25 g, 35.8 mmol) at RT. The reaction mixture was stirred overnight under nitrogen, then diluted with water (500 mL) and extracted with EtOAc (3×500 mL). The combined organic layers were washed with brine (500 mL), dried over anhydrous Na₂SO₄, filtered and concentrated under reduced pressure. The residue was purified on silica, eluting with 16-20% EtOAc in PE to obtain the title compound (5.0 g, 75%) as a yellow solid. MS (ES) m/z=329 (M+1).

### Preparation 10

### tert-Butyl 8-(6-bromo-3-(ethylthio)-5-fluoro-7,9-dihydrofuro[3,4-f]quinazolin-1-yl)-3,8-diazabicyclo[3.2.1]octane-3-carboxylate

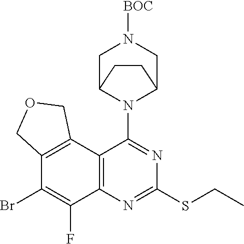 [SMILES: CCSc1nc(N2C3CCC2CN(C(=O)OC(C)(C)C)C3)c2c3c(c(Br)c(F)c2n1)COC3]

A suspension of 6-bromo-1-chloro-3-(ethylthio)-5-fluoro-7,9-dihydrofuro[3,4-f]quinazoline (21.0 g, 57.8 mmol) in ACN (580 mL) was charged with tert-butyl 3,8-diazabicyclo[3.2.1]octane-3-carboxylate (15.2 g, 69.5 mmol, 1.20 eq.) and DIPEA (40 mL, 229 mmol, 4 eq.) and was stirred at RT for 90 min. H₂O (1 L) was added slowly via addition funnel and the mixture was stirred at RT for 1 h. The solids were filtered, rinsed with H₂O (500 mL) and dried under vacuum at 50° C. to obtain the title compound (31 g, quantitative) as a white solid, MS (ES) m/z=539 (M+1).

### Preparation 11

### 6-Bromo-1-(3-((R)-2-((tert-butyldimethylsilyl)oxy)propyl)-3,8-diazabicyclo[3.2.1]octan-8-yl)-3-(ethylthio)-5-fluoro-7,9-dihydrofuro[3,4-f]quinazoline

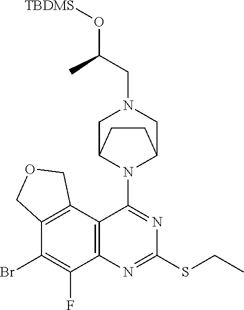

3-((R)-2-((tert-butyldimethylsilyl)oxy)propyl)-3,8-diazabicyclo[3.2.1]octane was used in a manner analogous to that described for Preparation 10 to obtain the title compound (4.7 g, 95%) as a tan solid.

### Preparation 12

### tert-Butyl (4-chloro-3-cyano-7-fluorothieno[3,2-c]pyridin-2-yl)carbamate

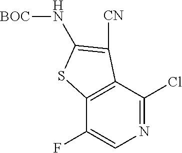 [SMILES: CC(C)(C)OC(=O)Nc1sc2c(F)cnc(Cl)c2c1C#N]

Ethyl (3-cyano-7-fluorothieno[3,2-c]pyridin-2-yl)carbamate. A solution of 2-(4-chloro-5-fluoropyridin-3-yl)acetonitrile (11.8 g, 56.1 mmol) in DMF (112 mL) was cooled to 0° C. Potassium tert-butoxide (7.00 g, 61.1 mmol) was added. After 15 min, ethoxycarbonyl isothiocyanate (7.45 mL, 61.8 mmol) was added dropwise. The reaction mixture was allowed to slowly warm to RT overnight. The reaction mixture was poured into a mixture of ice/water (1.5 L), stirred until all ice had melted, and filtered through diatomaceous earth. The solids were dried in a vacuum oven (60° C.) overnight and separated from the diatomaceous earth to give ethyl N-(3-cyano-7-fluoro-thieno[3,2-c]pyridin-2-yl)carbamate (11.9 g, 79%) as a solid. MS (ES) m/z=266 (M+1).

2-Amino-7-fluorothieno[3,2-c]pyridine-3-carbonitrile. A suspension of ethyl (3-cyano-7-fluorothieno[3,2-c]pyridin-2-yl)carbamate (11.9 g, 44.4 mmol) in DMSO (90 mL) was cooled to 0° C. NaOH (5 M in water, 90 mL) was added dropwise over 15 min. The reaction mixture was heated to 105° C. for 1 h, then cooled to RT. The reaction mixture was poured into a mixture of ice/water (1.8 L), stirred until all ice had melted, and filtered through diatomaceous earth. The solids were dried in a vacuum oven (50° C.) overnight and separated from the diatomaceous earth to give crude 2-amino-7-fluoro-thieno[3,2-c]pyridine-3-carbonitrile.

tert-Butyl (3-cyano-7-fluorothieno[3,2-c]pyridin-2-yl)carbamate. A mixture of crude 2-amino-7-fluorothieno[3,2-c]pyridine-3-carbonitrile (8.6 g, 44.4 mmol), DCM (90 mL), DMF (90 mL) and N,N-diisopropylethylamine (15.5 mL, 88.9 mmol) was cooled to 0° C. 4-dimethylaminopyridine (0.54 g, 4.42 mmol) and di-tert-butyl dicarbonate (14.6 g, 66.7 mmol) were added. The reaction mixture was stirred at RT for 2 h. The solvents were removed under reduced pressure and the remaining material was diluted with DCM (400 mL) and 5% aq. citric acid (250 mL). The aqueous phase was washed twice with DCM. The combined organic phases were washed with sat. aq. NaHCO₃, dried over MgSO₄, filtered, and concentrated to give tert-butyl N-(3-cyano-7-fluoro-thieno[3,2-c]pyridin-2-yl)carbamate (7.5 g, 58%) as a brown solid. MS (ES) m/z=294 (M+1).

2-((tert-Butoxycarbonyl)amino)-3-cyano-7-fluorothieno[3,2-c]pyridine 5-oxide. 3-Chloroperoxybenzoic acid (9.00 g, 40.2 mmol) was added to a solution of tert-butyl (3-cyano-7-fluorothieno[3,2-c]pyridin-2-yl)carbamate (7.85 g, 26.8 mmol) in DCM (180 mL). The reaction mixture was stirred at RT overnight, then cooled to 0° C. for −15 min. Solids were collected by filtration and dried in a vacuum oven (60° C.). The filtrate was diluted with MeOH and silica gel, concentrated, and the residue was purified on silica, eluting with 0-6% MeOH in DCM. Fractions containing desired material were combined with the solids from the filtration and concentrated to give tert-butyl N-(3-cyano-7-fluoro-5-oxido-thieno[3,2-c]pyridin-5-ium-2-yl)carbamate (7.26 g, 88%) as an off-white solid. MS (ES) m/z=310 (M+1).

tert-Butyl (4-chloro-3-cyano-7-fluorothieno[3,2-c]pyridin-2-yl)carbamate. A suspension of 2-((tert-butoxycarbonyl)amino)-3-cyano-7-fluorothieno[3,2-c]pyridine 5-oxide (5.27 g, 17.0 mmol) in 1,2-dichloroethane (34 mL) was cooled to 0° C. A solution of phosphoryl chloride (32 mL, 344 mmol) in 1,2-dichloroethane (34 mL) was added dropwise. The reaction mixture was stirred at RT for 30 min, at 45° C. for 90 min, and cooled to RT. The reaction mixture was diluted with 1,2-dichloroethane (100 mL) and added to a mixture of sat. aq. NaHCO₃ (500 mL), NaOH (5 M in water, 40 mL), and ice. Solid NaHCO₃ was added to the stirred mixture to maintain pH˜6-7. Once bubbling ceased, the phases were separated. The aqueous phase was extracted 3× with DCM. The combined organic phases were dried over MgSO₄ and filtered. The filtrate was diluted with MeOH and silica gel, concentrated, and the residue was purified on silica, eluting with 50-100% DCM in hexanes. Fractions containing desired material were concentrated to give the title compound (3.87 g, 69%) as a white solid. MS (ES) m/z=328 (M+1).

### Preparation 13

### tert-Butyl 8-(6-(2-((tert-butoxycarbonyl)amino)-3-cyano-7-fluorothieno[3,2-c]pyridin-4-yl)-3-(ethylthio)-5-fluoro-7,9-dihydrofuro[3,4-f]quinazolin-1-yl)-3,8-diazabicyclo[3.2.1]octane-3-carboxylate

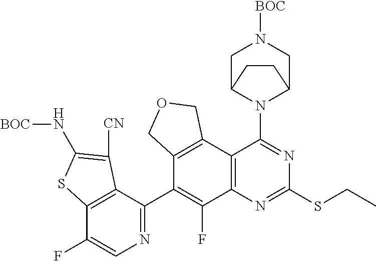 [SMILES: CCSc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3]

To a solution of tert-butyl 8-(6-bromo-3-(ethylthio)-5-fluoro-7,9-dihydrofuro[3,4-f]quinazolin-1-yl)-3,8-diazabicyclo[3.2.1]octane-3-carboxylate (25.0 g, 46.3 mmol) and 5,5,5′,5′-tetramethyl-2,2′-bi(1,3,2-dioxaborinane) (20.9 g, 92.7 mmol) in 1,4-dioxane (300 mL) was added potassium acetate (13.6 g, 139 mmol) and dichloropalladium; {2-[2-(diphenylphosphanyl)phenoxy]phenyl}diphenylphosphane (4.98 g, 6.95 mmol) at RT under nitrogen. The mixture was stirred overnight at 85° C., then filtered. The filter cake was washed with 1,4-dioxane (2×100 mL). The filtrate was concentrated under reduced pressure and purified on silica, eluting with 0-20% EtOAc in PE to obtain tert-butyl 8-(6-(5,5-dimethyl-1,3,2-dioxaborinan-2-yl)-3-(ethylthio)-5-fluoro-7,9-dihydrofuro[3,4-f]quinazolin-1-yl)-3,8-diazabicyclo[3.2.1]octane-3-carboxylate (23 g, 87%) as a yellow solid.

To a stirred mixture of tert-butyl 8-(6-(5,5-dimethyl-1,3,2-dioxaborinan-2-yl)-3-(ethylthio)-5-fluoro-7,9-dihydrofuro[3,4-f]quinazolin-1-yl)-3,8-diazabicyclo[3.2.1]octane-3-carboxylate (6.99 g, 12.2 mmol) and tert-butyl (4-chloro-3-cyano-7-fluorothieno[3,2-c]pyridin-2-yl)carbamate (4.00 g, 12.2 mmol) in toluene (150 mL) was added potassium phosphate (7.77 g, 36.6 mmol), [2-dicyclohexylphosphino-2′,4′,6′-triisopropylbiphenyl](1.75 g, 3.66 mmol), and XPhos Palladacycle Gen 4 (CAS 1599466-81-5; 0.026 g, 0.031 mmol) at RT under nitrogen. The mixture was stirred for 4 h at 80° C., then diluted with water (300 mL) and extracted with EtOAc (3×200 mL). The combined organic layers were washed with brine (2×300 mL), dried over anhydrous Na₂SO₄, filtered and concentrated under reduced pressure. The residue was purified on silica, eluting with 0-45% EtOAc in PE to obtain the title compound (6.0 g, 65%) as a yellow solid. MS (ES) m/z=752 (M+1).

The following compounds in Table 1 were prepared in similar manner as described in Preparation 13. Various methods were used to purify the compounds, which would be apparent to one skilled in the art.

TABLE 1

| | | | MS |
|---|---|---|---|
| | | | (ES) m/z |
| Preparation | Chemical Name | Structure | (M + 1) |
| 14 | tert-Butyl (3-cyano-4-(3- (ethylthio)-5-fluoro-7,9- dihydrofuro[3,4- f]quinazolin-6-yl)-7- fluorothieno[3,2- c]pyridin-2-yl)carbamate | 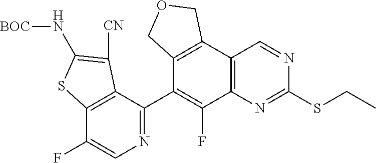 [SMILES: CCSc1ncc2c3c(c(-c4ncc(F)c5sc(NC(=O)OC(C)(C)C)c(C#N)c45)c(F)c2n1)COC3] | 542 |
| 15 | tert-Butyl (4-(1-(3-((R)- 2-((tert- butyldimethylsilyl)oxy)pro- pyl)-3,8- diazabicyclo[3.2.1]octan- 8-yl)-3-(ethylthio)-5- fluoro-7,9- dihydrofuro[3,4- f]quinazolin-6-yl)-3- cyano-7-fluorothieno[3,2- c]pyridin-2-yl)carbamate | 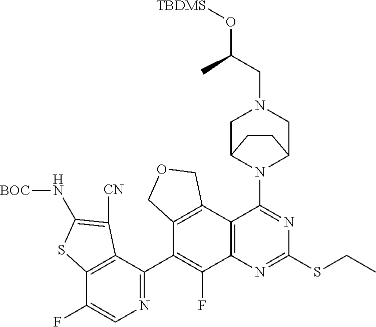 | 824 |

### Preparation 16

### tert-Butyl 8-(6-(2-((tert-butoxycarbonyl)amino)-3-cyano-7-fluorothieno[3,2-c]pyridin-4-yl)-3-(ethyl sulfonyl)-5-fluoro-7,9-dihydrofuro[3,4-f]quinazolin-1-yl)-3,8-diazabicyclo[3.2.1]octane-3-carboxylate

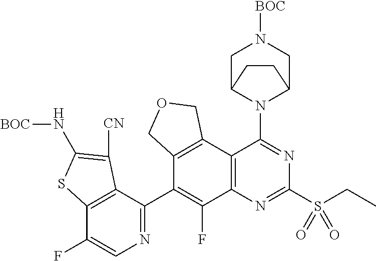 [SMILES: CCS(=O)(=O)c1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3]

To a solution of tert-butyl 8-(6-(2-((tert-butoxycarbonyl)amino)-3-cyano-7-fluorothieno[3,2-c]pyridin-4-yl)-3-(ethylthio)-5-fluoro-7,9-dihydrofuro[3,4-f]quinazolin-1-yl)-3,8-diazabicyclo[3.2.1]octane-3-carboxylate (8.4 g, 11.2 mmol) in DCM (100 mL) was added mCPBA (4.05 g, 23.5 mmol) in portions at 0° C. The reaction mixture was stirred for 3 h at 0° C. The mixture was diluted with DCM (800 mL) and washed with sat. aq. NaHCO₃ (3×400 mL) and brine (2×300 mL). The organics were dried over anhydrous Na₂SO₄, filtered and concentrated under reduced pressure. The residue was purified on silica, eluting with 0-10% MeOH in DCM to obtain the title compound (8.3 g, 95%) as an off-white solid. MS (ES) m/z=784 (M+1).

The following compounds in Table 2 were prepared in similar manner as described in Preparation 16. Various methods were used to purify the compounds, which would be apparent to one skilled in the art.

TABLE 2

| | | | MS |
|---|---|---|---|
| | | | (ES) m/z |
| Preparation | Chemical Name | Structure | (M + 1) |
| 17 | tert-Butyl (3-cyano-4-(3- (ethylsulfonyl)-5-fluoro- 7,9-dihydrofuro[3,4- f]quinazolin-6-yl)-7- fluorothieno[3,2- c]pyridin-2-yl)carbamate | 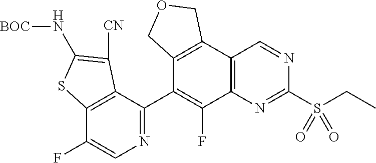 [SMILES: CCS(=O)(=O)c1ncc2c3c(c(-c4ncc(F)c5sc(NC(=O)OC(C)(C)C)c(C#N)c45)c(F)c2n1)COC3] | 574 |

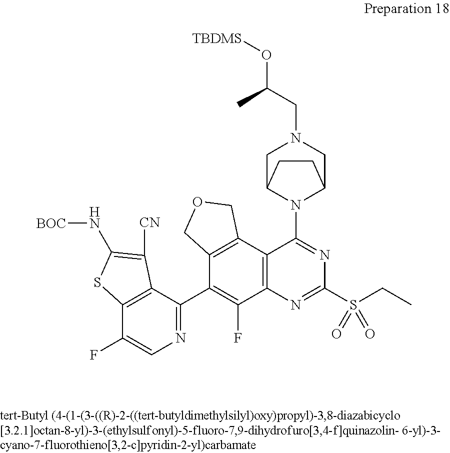

tert-Butyl (4-(1-(3-((R)-2-((tert-butyldimethylsilyl)oxy)propyl)-3,8-diazabicyclo[3.2.1]octan-8-yl)-3-(ethylthio)-5-fluoro-7,9-dihydrofuro[3,4-f]quinazolin-6-yl)-3-cyano-7-fluorothieno[3,2-c]pyridin-2-yl)carbamate (4 g, 5 mmol), hexaammonium heptamolybdate tetrahydrate (1 g, 1 mmol), and hydrogen peroxide (8 mL, 35 wt % in water, 100 mmol) were combined in DCM (30 mL) and EtOH (30 mL). The mixture was stirred at RT for 1 h, then concentrated under reduced pressure. Diluted with water (100 mL) and stirred 30 min. Filtered and dried the resulting solids in a vacuum oven (45° C.) to give the title compound (2 g, 50%) as a white solid. MS (ES) m/z=856 (M+1).

### Preparation 19

### tert-Butyl (2R,3S)-3-fluoro-2-(hydroxymethyl)pyrrolidine-1-carboxylate

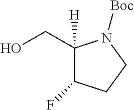 [SMILES: CC(C)(C)OC(=O)N1CC[C@H](F)[C@H]1CO]

A solution of 1-(tert-butyl) 2-methyl (2R,3S)-3-fluoropyrrolidine-1,2-dicarboxylate (1.02 g, 4.13 mmol) in THE (20 mL) was treated with lithium chloride (0.385 g, 9.08 mmol) and sodium borohydride (0.390 g, 10.3 mmol). The mixture was cooled to 0° C. and ethanol (40 mL) was added dropwise. The reaction mixture was allowed to slowly warm to RT and stirred overnight. The mixture was cooled to 0° C., acidified to pH˜4 with 1M aqueous citric acid, and concentrated under reduced pressure to remove volatiles. The remaining material was diluted with water and extracted with EtOAc (3×). The combined organics were dried over anhydrous Na₂SO₄, filtered, and concentrated under reduced pressure to obtain the title compound (0.95 g, 100%) as a colorless oil. MS (ES) m/z=164 (M+1, -tBu).

The following compounds in Table 3 were prepared in similar manner as described in Preparation 19. Various methods were used to purify the compounds, which would be apparent to one skilled in the art.

TABLE 3

| | | | MS |
|---|---|---|---|
| | | | (ES) m/z |
| Preparation | Chemical Name | Structure | (M + 1) |
| 20 | tert-Butyl (2S,4R)-4- fluoro-2- (hydroxymethyl)pyrrolidine- 1-carboxylate | 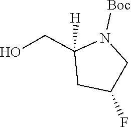 [SMILES: CC(C)(C)OC(=O)N1C[C@H](F)C[C@H]1CO] | 164 (-tBu) |

### Preparation 21

### tert-Butyl (2R,3S)-3-fluoro-2-formylpyrrolidine-1-carboxylate

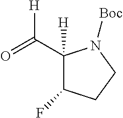 [SMILES: CC(C)(C)OC(=O)N1CC[C@H](F)[C@H]1C=O]

A 0° C. mixture of tert-butyl (2R,3S)-3-fluoro-2-(hydroxymethyl)pyrrolidine-1-carboxylate (1.96 g, 8.94 mmol) in DCM (50 mL) was treated with Dess-Martin periodinane (4.74 g, 11.2 mmol). The reaction mixture was allowed to slowly warm to RT and stirred overnight. The mixture was cooled to 0° C., treated cautiously with saturated aqueous sodium bicarbonate, and diluted with water. The organic layer was separated. The aqueous layer (pH˜8) was extracted with DCM (3×). The combined organics were dried over anhydrous Na₂SO₄, filtered, and concentrated under reduced pressure. The residue was purified on silica, eluting with 0-40% EtOAc in hexanes to obtain the title compound (1.22 g, 63%) as a light yellow oil. MS (ES) m/z=162 (M+1, -tBu).

The following compounds in Table 4 were prepared in similar manner as described in Preparation 21. Various methods were used to purify the compounds, which would be apparent to one skilled in the art.

TABLE 4

| | | | MS |
|---|---|---|---|
| | | | (ES) m/z |
| Preparation | Chemical Name | Structure | (M + 1) |
| 22 | tert-Butyl (2S,4R)-4- fluoro-2- formylpyrrolidine-1- carboxylate | 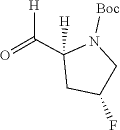 [SMILES: CC(C)(C)OC(=O)N1C[C@H](F)C[C@H]1C=O] | 162 (-tBu) |

### Preparation 23

### tert-Butyl (2S,4R)-2-formyl-4-methoxypyrrolidine-1-carboxylate

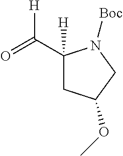 [SMILES: CO[C@@H]1C[C@@H](C=O)N(C(=O)OC(C)(C)C)C1]

A −75° C. mixture of 1-(tert-butyl) 2-methyl (2S,4R)-4-methoxypyrrolidine-1,2-dicarboxylate (9.84 g, 37.9 mmol) in DCM (190 mL) was treated with dropwise addition of diisobutylaluminium hydride (1M in DCM; 79.7 mL, 79.7 mmol). The reaction mixture was stirred at −75° C. for 2.5 h, diluted with MeOH (10 mL), and allowed to warm to RT. The mixture was treated with dropwise addition of saturated aqueous potassium sodium tartrate (100 mL), stirred at RT for 1 h, and extracted with DCM (2×250 mL). The combined organics were washed with brine, dried over magnesium sulfate, filtered, and concentrated under reduced pressure. The residue was purified on silica, eluting with 0-60% EtOAc in hexanes to obtain the title compound (8.00 g, 92%).

### Preparations 24 and 25

### tert-Butyl (2R,3S)-3-fluoro-2-(1-hydroxyethyl)pyrrolidine-1-carboxylate, Isomers 1 and 2

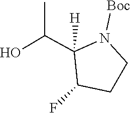 [SMILES: CC(O)[C@@H]1[C@@H](F)CCN1C(=O)OC(C)(C)C]

A −70° C. solution of tert-butyl (2R,3S)-3-fluoro-2-formylpyrrolidine-1-carboxylate (1.22 g, 5.62 mmol) in THF (30 mL) was treated with methylmagnesium bromide (1.4M in 3:1 THF:toluene; 6.0 mL, 8.4 mmol), maintaining the internal temperature below −60° C. The reaction mixture was stirred for 1 h, then allowed to warm to 0° C. The reaction mixture was cooled to −30° C. and treated with additional methylmagnesium bromide (1.4M in 3:1 THF:toluene; 2.0 mL, 2.8 mmol). The reaction mixture was stirred for 1 h, then allowed to warm to 15° C. The reaction mixture was cooled to 0° C. and treated with acetic acid (1.29 mL, 22.5 mmol). The reaction mixture was stirred at RT for 15 min, diluted with saturated aqueous ammonium chloride and EtOAc, and stirred for 5 min. A small amount of water was added. The organic layer was separated. The aqueous layer (pH˜5) was extracted with EtOAc (3×). The combined organics were dried over anhydrous Na₂SO₄, filtered, and concentrated under reduced pressure. The residue was purified on silica, eluting with 0-10% MTBE in DCM to obtain the title compounds (Isomer 1 was first eluting, 0.279 g, 21%; Isomer 2 was second eluting, 0.297 g, 23%) as white solids. MS (ES) m/z=178 (M+1, -tBu) for both.

The following compounds in Table 5 were prepared in similar manner as described in Preparations 24 and 25. Various methods were used to purify the compounds, which would be apparent to one skilled in the art.

TABLE 5

| | | | MS |
|---|---|---|---|
| | | | (ES) m/z |
| Preparation | Chemical Name | Structure | (M + 1) |
| 26¹ | tert-Butyl (2S,4R)-4- fluoro-2-(1- hydroxyethyl)pyrrolidine- 1-carboxylate, Isomer 1 | 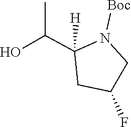 [SMILES: CC(O)[C@@H]1C[C@@H](F)CN1C(=O)OC(C)(C)C] | 178 (-tBu) |
| 27¹ | tert-Butyl (2S,4R)-4- fluoro-2-(1- hydroxyethyl)pyrrolidine- 1-carboxylate, Isomer 2 | 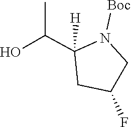 [SMILES: CC(O)[C@@H]1C[C@@H](F)CN1C(=O)OC(C)(C)C] | 178 (-tBu) |
| 28² | tert-Butyl (2S,4R)-2-(1- hydroxyethyl)-4- methoxypyrrolidine-1- carboxylate, Isomer 1 | 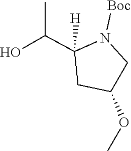 [SMILES: CO[C@@H]1C[C@@H](C(C)O)N(C(=O)OC(C)(C)C)C1] | |

¹Silica purification; 5% THF in DCM
²Silica purification; 2-10% THF in DCM

### Preparation 29

### 1-((2R,3S)-3-Fluoro-1-methylpyrrolidin-2-yl)ethan-1-ol, Isomer 1

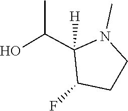

A 0° C. mixture of tert-butyl (2R,3S)-3-fluoro-2-(1-hydroxyethyl)pyrrolidine-1-carboxylate, Isomer 1 (single methyl isomer from precursor in Preparation 24; 0.279 g, 1.20 mmol) in THE (10 mL) was treated with lithium aluminum hydride (1.M in THF; 3.59 mL, 3.59 mmol). The reaction mixture was heated at 70° C. overnight, cooled to 0° C., and quenched with sodium sulfate decahydrate (1.19 g, 3.69 mmol). The mixture was diluted with diethyl ether (10 mL), allowed to warm to RT, and stirred for 2 h. The mixture was filtered through diatomaceous earth and rinsed with diethyl ether. The combined filtrates were concentrated under reduced pressure. The residue was purified on silica, eluting with 0-5% 1M ammoniated methanol in DCM to obtain the title compound (0.092 g, 520) as a yellow oil. MS (ES) m/z=148 (M+1).

The following compounds in Table 6 were prepared in similar manner as described in Preparation 29. Various methods were used to purify the compounds, which would be apparent to one skilled in the art.

TABLE 6

| | | | MS |
|---|---|---|---|
| | | | (ES) m/z |
| Preparation | Chemical Name | Structure | (M + 1) |
| 30¹ | 1-((2S,4R)-4-Fluoro-1- methylpyrrolidin-2- yl)ethan-1-ol, Isomer 1 | 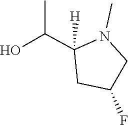 | 148 |
| 31² | 1-((2S,4R)-4-Fluoro-1- methylpyrrolidin-2- yl)ethan-1-ol, Isomer 2 | 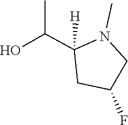 | 148 |
| 32³ | 1-((2R,3S)-3-Fluoro-1- methylpyrrolidin-2- yl)ethan-1-ol, Isomer 2 | 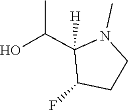 | 148 |
| 33⁴ | 1-((2S,4R)-4-Methoxy-1- methylpyrrolidin-2- yl)ethan-1-ol, Isomer 1 | 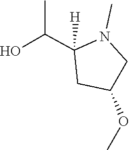 | 160 |

¹Single methyl isomer (from precursor in Preparation 26)
²Single methyl isomer (from precursor in Preparation 27)
³Single methyl isomer (from precursor in Preparation 25)
⁴Single methyl isomer (from precursor in Preparation 28)

### Preparation 34

### tert-Butyl 8-(6-(2-((tert-butoxycarbonyl)amino)-3-cyano-7-fluorothieno[3,2-c]pyridin-4-yl)-5-fluoro-3-(1-((2R,3S)-3-fluoro-1-methylpyrrolidin-2-yl)ethoxy)-7,9-dihydrofuro[3,4-f]quinazolin-1-yl)-3,8-diazabicyclo[3.2.1]octane-3-carboxylate, Isomer 1

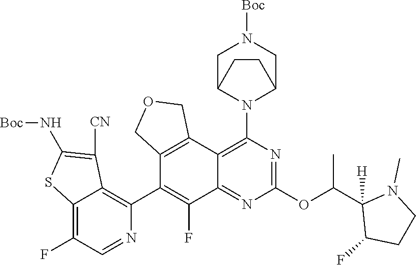 [SMILES: CC(Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1[C@@H](F)CCN1C]

A solution of 1-((2R,3S)-3-fluoro-1-methylpyrrolidin-2-yl)ethan-1-ol, Isomer 1 (single methyl isomer from precursor in Preparation 24; 0.090 g, 0.61 mmol) in THE (5 mL) was treated with lithium bis(trimethylsilyl)amide (1.M in THF; 1.3 mL, 1.3 mmol). The reaction mixture was stirred for 10 min and tert-butyl 8-(6-(2-((tert-butoxycarbonyl)amino)-3-cyano-7-fluorothieno[3,2-c]pyridin-4-yl)-3-(ethylsulfonyl)-5-fluoro-7,9-dihydrofuro[3,4-f]quinazolin-1-yl)-3,8-diazabicyclo[3.2.1]octane-3-carboxylate (0.400 g, 0.51 mmol) was added. The reaction mixture was stirred for 1 h and lithium bis(trimethylsilyl)amide (1.M in THF; 1.3 mL, 1.3 mmol) was added. The reaction mixture was stirred for 1 h, then diluted with saturated aqueous ammonium chloride and EtOAc. A small amount of water was added. The organic layer was separated. The aqueous layer (pH˜7-8) was extracted with EtOAc (2×). The combined organics were dried over anhydrous Na₂SO₄, filtered, and concentrated under reduced pressure. The residue was purified on silica, eluting with 0-5% 1M ammoniated methanol in DCM to obtain the title compound (0.168 g, 39%) as an orange solid. MS (ES) m/z=837 (M+1).

The following compounds in Table 7 were prepared in similar manner as described in Preparation 34. Various methods were used to purify the compounds, which would be apparent to one skilled in the art.

TABLE 7

| | | | MS |
|---|---|---|---|
| | | | (ES) m/z |
| Preparation | Chemical Name | Structure | (M + 1) |
| 35¹ | tert-Butyl 8-(6-(2-((tert- butoxycarbonyl)amino)- 3-cyano-7- fluorothieno[3,2- c]pyridin-4-yl)-5-fluoro- 3-(1-((2S,4R)-4-fluoro-1- methylpyrrolidin-2- yl)ethoxy)-7,9- dihydrofuro[3,4- f]quinazolin-1-yl)-3,8- diazabicyclo[3.2.1]octane- 3-carboxylate, Isomer 1 | 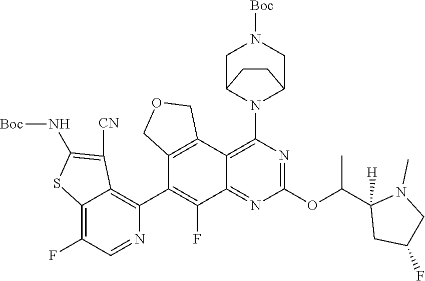 [SMILES: CC(Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1C[C@@H](F)CN1C] | 837 |
| 36² | tert-Butyl 8-(6-(2-((tert- butoxycarbonyl)amino)- 3-cyano-7- fluorothieno[3,2- c]pyridin-4-yl)-5-fluoro- 3-(1-((2S,4R)-4-fluoro-1- methylpyrrolidin-2- yl)ethoxy)-7,9- dihydrofuro[3,4- f]quinazolin-1-yl)-3,8- diazabicyclo[3.2.1]octane- 3-carboxylate, Isomer 2 | 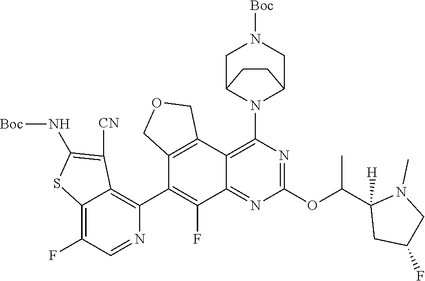 [SMILES: CC(Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1C[C@@H](F)CN1C] | 837 |
| 37³ | tert-Butyl 8-(6-(2-((tert- butoxycarbonyl)amino)- 3-cyano-7- fluroothieno[3,2- c]pyridin-4-yl)-5-fluoro- 3-(1-((2R,3S)-3-fluoro-1- methylpyrrolidin-2- yl)ethoxy)-7,9- dihydrofuro[3,4- f]quinazolin-1-yl)-3,8- diazabicyclo[3.2.1]octane- 3-carboxylate, Isomer 2 | 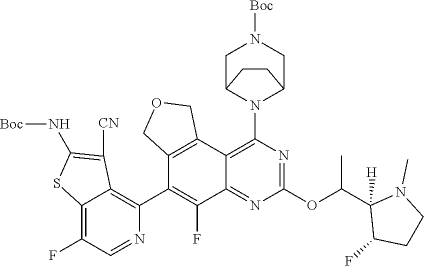 [SMILES: CC(Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1[C@@H](F)CCN1C] | 837 |
| 38⁴ | tert-Butyl 8-(6-(2-((tert- butoxycarbonyl)amino)- 3-cyano-7- fluorothieno[3,2- c]pyridin-4-yl)-5-fluoro- 3-(1-((2S,4R)-4- methoxy-1- methylpyrrolidin-2- yl)ethoxy)-7,9- dihydrofuro[3,4- f]quinazolin-1-yl)-3,8- diazabicyclo[3.2.1]octane- 3-carboxylate, Isomer 1 | 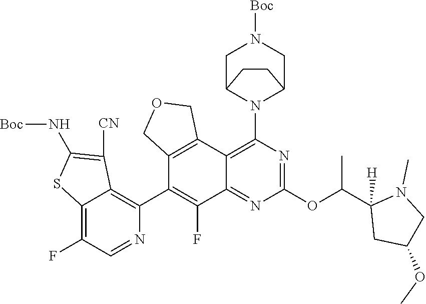 [SMILES: CO[C@@H]1C[C@@H](C(C)Oc2nc(N3C4CCC3CN(C(=O)OC(C)(C)C)C4)c3c4c(c(-c5ncc(F)c6sc(NC(=O)OC(C)(C)C)c(C#N)c56)c(F)c3n2)COC4)N(C)C1] | 849 |
| 39 | tert-Butyl (4-(1-(3-((R)- 2-((tert- butyldimethylsilyl)oxy)pro- pyl)-3,8- diazabicyclo[3.2.1]octan- 8-yl)-5-fluoro-3-((S)-1- ((S)-1-methylpyrrolidin- 2-yl)ethoxy)-7,9- dihydrofuro[3,4- f]quinazolin-6-yl)-3- cyano-7-fluorothieno[3,2- c]pyridin-2-yl)carbamate | 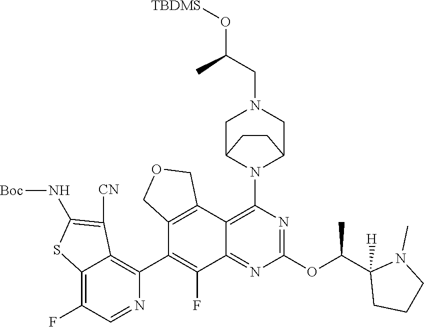 | 891 |
| 40 | tert-Butyl 8-(6-(2-((tert- butoxycarbonyl)amino)- 3-cyano-7- fluorothieno[3,2- c]pyridin-4-yl)-5-fluoro- 3-((S)-1-((S)-1- methylpyrrolidin-2- yl)ethoxy)-7,9- dihydrofuro[3,4- f]quinazolin-1-yl)-3,8- diazabicyclo[3.2.1]octane- 3-carboxylate | 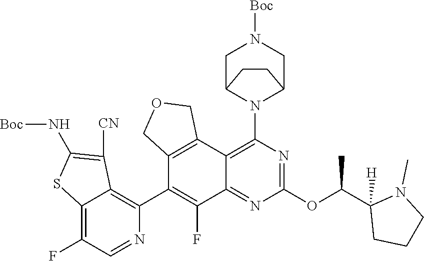 [SMILES: C[C@H](Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1CCCN1C] | 819 |
| 41 | tert-Butyl 8-(6-(2-((tert- butoxycarbonyl)amino)- 3-cyano-7- fluorothieno[3,2- c]pyridin-4-yl)-5-fluoro- 3-((R)-1-((S)-1- methylpyrrolidin-2- yl)ethoxy)-7,9- dihydrofuro[3,4- f]quinazolin-1-yl)-3,8- diazabicyclo[3.2.1]octane- 3-carboxylate | 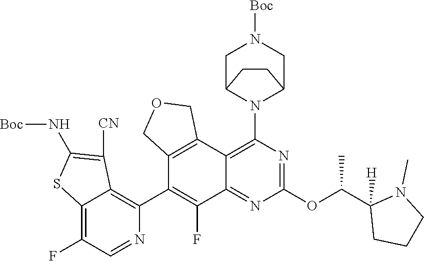 [SMILES: C[C@@H](Oc1nc(N2C3CCC2CN(C(=O)OC(C)(C)C)C3)c2c3c(c(-c4ncc(F)c5sc(NC(=O)OC(C)(C)C)c(C#N)c45)c(F)c2n1)COC3)[C@@H]1CCCN1C] | 819 |
| 42 | tert-Butyl (3-cyano-7- fluoro-4-(5-fluoro-3-((S)- 1-((S)-1- methylpyrrolidin-2- yl)ethoxy)-7,9- dihydrofuro[3,4- f]quinazolin-6- yl)thieno[3,2-c]pyridin-2- yl)carbamate | 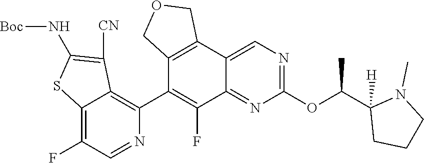 [SMILES: C[C@H](Oc1ncc2c3c(c(-c4ncc(F)c5sc(NC(=O)OC(C)(C)C)c(C#N)c45)c(F)c2n1)COC3)[C@@H]1CCCN1C] | 609 |
| 43⁴ | tert-Butyl (3-cyano-7- fluoro-4-(5-fluoro-3-(1- ((2S,4R)-4-methoxy-1- methylpyrrolidin-2- yl)ethoxy)-7,9- dihydrofuro[3,4- f]quinazolin-6- yl)thieno[3,2-c]pyridin-2- yl)carbamate | 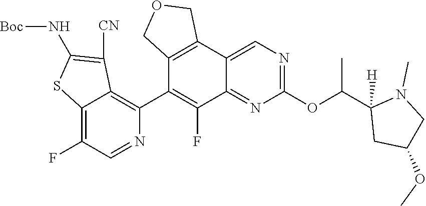 [SMILES: CO[C@@H]1C[C@@H](C(C)Oc2ncc3c4c(c(-c5ncc(F)c6sc(NC(=O)OC(C)(C)C)c(C#N)c56)c(F)c3n2)COC4)N(C)C1] | 639 |

¹Single methyl isomer (from precursor in Preparation 26)
²Single methyl isomer (from precursor in Preparation 27)
³Single methyl isomer (from precursor in Preparation 25)
⁴Single methyl isomer (from precursor in Preparation 28)

### Preparation 44

### 4-(1-(3,8-Diazabicyclo[3.2.1]octan-8-yl)-5-fluoro-3-(1-((2R,3S)-3-fluoro-1-methylpyrrolidin-2-yl)ethoxy)-7,9-dihydrofuro[3,4-f]quinazolin-6-yl)-2-amino-7-fluorothieno[3,2-c]pyridine-3-carbonitrile, Isomer 1

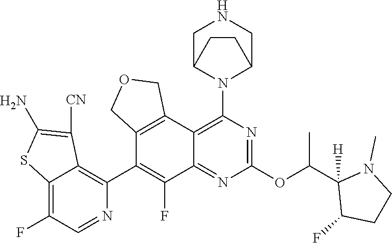 [SMILES: CC(Oc1nc(N2C3CCC2CNC3)c2c3c(c(-c4ncc(F)c5sc(N)c(C#N)c45)c(F)c2n1)COC3)[C@@H]1[C@@H](F)CCN1C]

A solution of tert-butyl 8-(6-(2-((tert-butoxycarbonyl)amino)-3-cyano-7-fluorothieno[3,2-c]pyridin-4-yl)-5-fluoro-3-(1-((2R,3S)-3-fluoro-1-methylpyrrolidin-2-yl)ethoxy)-7,9-dihydrofuro[3,4-f]quinazolin-1-yl)-3,8-diazabicyclo[3.2.1]octane-3-carboxylate, Isomer 1 (single methyl isomer from precursor in Preparation 24; 0.165 g, 0.168 mmol) in DCM (2 mL) was treated with TFA (0.80 mL). The reaction mixture was stirred at RT overnight in a sealed vial with a nitrogen atmosphere, then concentrated under a stream of nitrogen. The residue was diluted with DCM, then concentrated under a stream of nitrogen. The residue was dissolved in MeOH and purified on strong cation exchange media (10 g), eluting first with MeOH and DCM, then with 1:1 2M ammoniated MeOH:DCM. The basic fraction was concentrated under reduced pressure. The residue was purified on silica, eluting with 0-13% 1M ammoniated methanol in DCM to obtain the title compound (0.078 g, 73%) as an orange solid. MS (ES) m/z=637 (M+1).

The following compounds in Table 8 were prepared in similar manner as described in Preparation 44. Various methods were used to purify the compounds, which would be apparent to one skilled in the art.

TABLE 8

| | | | MS |
|---|---|---|---|
| | | | (ES) m/z |
| Preparation | Chemical Name | Structure | (M + 1) |
| 45¹ | 4-(1-(3,8- Diazabicyclo[3.2.1]octan- 8-yl)-5-fluoro-3-(1- ((2S,4R)-4-fluoro-1- methylpyrrolidin-2- yl)ethoxy)-7,9- dihydrofuro[3,4- f]quinazolin-6-yl)-2- amino-7- fluorothieno[3,2- c]pyridine-3-carbonitrile, Isomer 1 | 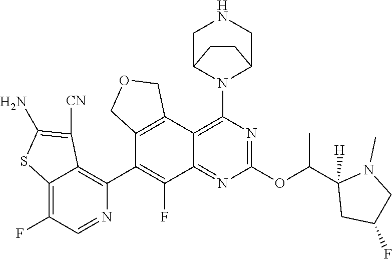 [SMILES: CC(Oc1nc(N2C3CCC2CNC3)c2c3c(c(-c4ncc(F)c5sc(N)c(C#N)c45)c(F)c2n1)COC3)[C@@H]1C[C@@H](F)CN1C] | 637 |
| 46² | 4-(1-(3,8- Diazabicyclo[3.2.1]octan- 8-yl)-5-fluoro-3-(1- ((2S,4R)-4-fluoro-1- methylpyrrolidin-2- yl)ethoxy)-7,9- dihydrofuro[3,4- f]quinazolin-6-yl)-2- amino-7- fluorothieno[3,2- c]pyridine-3-carbonitrile, Isomer 2 | 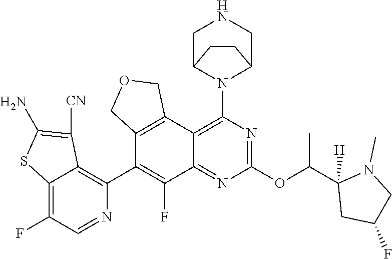 [SMILES: CC(Oc1nc(N2C3CCC2CNC3)c2c3c(c(-c4ncc(F)c5sc(N)c(C#N)c45)c(F)c2n1)COC3)[C@@H]1C[C@@H](F)CN1C] | 637 |
| 47³ | 4-(1-(3,8- Diazabicyclo[3.2.1]octan- 8-yl)-5-fluoro-3-(1- ((2R,3S)-3-fluoro-1- methylpyrrolidin-2- yl)ethoxy)-7,9- dihydrofuro[3,4- f]quinazolin-6-yl)-2- amino-7- fluorothieno[3,2- c]pyridine-3-carbonitrile, Isomer 2 | 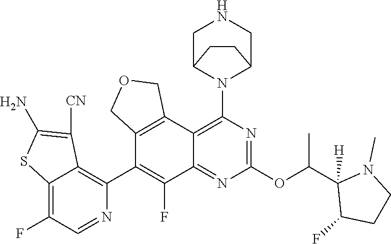 [SMILES: CC(Oc1nc(N2C3CCC2CNC3)c2c3c(c(-c4ncc(F)c5sc(N)c(C#N)c45)c(F)c2n1)COC3)[C@@H]1[C@@H](F)CCN1C] | 637 |
| 48⁴ | 4-(1-(3,8- Diazabicyclo[3.2.1]octan- 8-yl)-5-fluoro-3-(1- ((2S,4R)-4-methoxy-1- methylpyrrolidin-2- yl)ethoxy)-7,9- dihydrofuro[3,4- f]quinazolin-6-yl)-2- amino-7- fluorothieno[3,2- c]pyridine-3-carbonitrile, Isomer 1 | 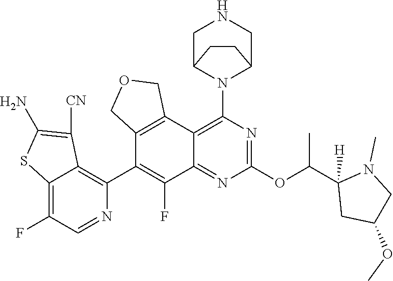 [SMILES: CO[C@@H]1C[C@@H](C(C)Oc2nc(N3C4CCC3CNC4)c3c4c(c(-c5ncc(F)c6sc(N)c(C#N)c56)c(F)c3n2)COC4)N(C)C1] | 649 |
| 49 | 4-(1-(3,8- Diazabicyclo[3.2.1]octan- 8-yl)-5-fluoro-3-((S)-1- ((S)-1-methylpyrrolidin- 2-yl)ethoxy)-7,9- dihydrofuro[3,4- f]quinazolin-6-yl)-2- amino-7- fluorothieno[3,2- c]pyridine-3-carbonitrile | 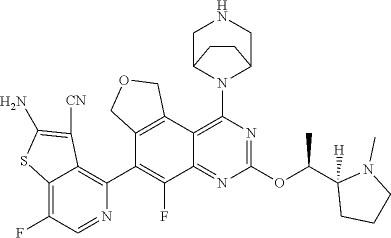 [SMILES: C[C@H](Oc1nc(N2C3CCC2CNC3)c2c3c(c(-c4ncc(F)c5sc(N)c(C#N)c45)c(F)c2n1)COC3)[C@@H]1CCCN1C] | 619 |
| 50 | 4-(1-(3,8- Diazabicyclo[3.2.1]octan- 8-yl)-5-fluoro-3-((R)-1- ((S)-1-methylpyrrolidin- 2-yl)ethoxy)-7,9- dihydrofuro[3,4- f]quinazolin-6-yl)-2- amino-7- fluorothieno[3,2- c]pyridine-3-carbonitrile | 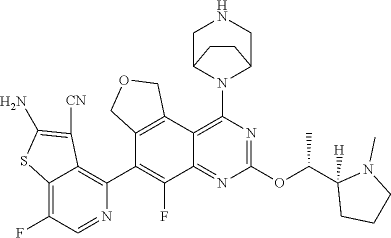 [SMILES: C[C@@H](Oc1nc(N2C3CCC2CNC3)c2c3c(c(-c4ncc(F)c5sc(N)c(C#N)c45)c(F)c2n1)COC3)[C@@H]1CCCN1C] | 619 |

¹Single methyl isomer (from precursor in Preparation 26)
²Single methyl isomer (from precursor in Preparation 27)
³Single methyl isomer (from precursor in Preparation 25)
⁴Single methyl isomer (from precursor in Preparation 28)

### Example 1

### 2-Amino-7-fluoro-4-(5-fluoro-3-(1-((2R,3S)-3-fluoro-1-methylpyrrolidin-2-yl)ethoxy)-1-(3-((R)-2-hydroxypropyl)-3,8-diazabicyclo[3.2.1]octan-8-yl)-7,9-dihydrofuro[3,4-f]quinazolin-6-yl)thieno[3,2-c]pyridine-3-carbonitrile, Isomer 1

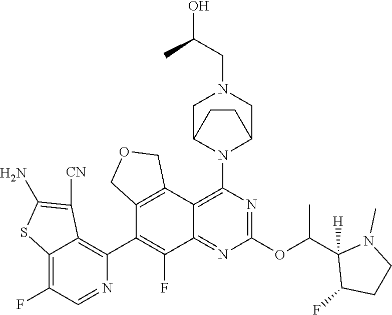 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1[C@@H](F)CCN1C]

A solution of 4-(1-(3,8-diazabicyclo[3.2.1]octan-8-yl)-5-fluoro-3-(1-((2R,3S)— 3-fluoro-1-methylpyrrolidin-2-yl)ethoxy)-7,9-dihydrofuro[3,4-f]quinazolin-6-yl)-2-amino-7-fluorothieno[3,2-c]pyridine-3-carbonitrile, Isomer 1 (single methyl isomer from precursor in Preparation 24; 0.075 g, 0.12 mmol) in THE (1.2 mL) and MeOH (0.3 mL) was treated with sodium triacetoxyborohydride (0.38 g, 1.8 mmol) and (R)-2-hydroxypropanal (0.9M in water; 2.0 mL, 1.8 mmol). The reaction mixture was stirred at RT for 1 h, then poured into ice cold saturated aqueous sodium bicarbonate. The organic layer was separated. The aqueous layer (pH˜8) was extracted with DCM (2×). The combined organics were passed through a hydrophobic frit and concentrated under a stream of nitrogen. The residue was purified on silica, eluting with 0-7.5% 1M ammoniated methanol in DCM to obtain the title compound (0.048 g, 59%) as an off-white solid. MS (ES) m/z=695 (M+1).

The following compounds in Table 9 were prepared in similar manner as described in Example 1. Various methods were used to purify the compounds, which would be apparent to one skilled in the art.

TABLE 9

| | | | MS |
|---|---|---|---|
| | | | (ES) m/z |
| Example | Chemical Name | Structure | (M + 1) |
| 2¹ | 2-Amino-7-fluoro-4-(5- fluoro-3-(1-((2S,4R)-4- fluoro-1- methylpyrrolidin-2- yl)ethoxy)-1-(3-((R)-2- hydroxypropyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 1 | 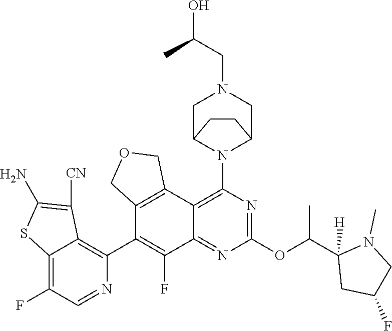 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1C[C@@H](F)CN1C] | 695 |
| 3² | 2-Amino-7-fluoro-4-(5- fluoro-3-(1-((2S,4R)-4- fluoro-1- methylpyrrolidin-2- yl)ethoxy)-1-(3-((R)-2- hydroxypropyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 2 | 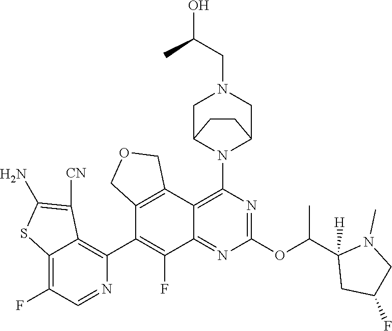 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1C[C@@H](F)CN1C] | 695 |
| 4¹ | 2-Amino-7-fluoro-4-(5- fluoro-3-(1-((2S,4R)-4- fluoro-1- methypyrrolidin-2- yl)ethoxy)-1-(3-methyl- 3,8- diazabicyclo[3.2.1]octan- 8-yl)-7,9- dihydrofuro[3,4- f]quinazolin-6- yl)thieno[3,2-c]pyridine- 3-carbonitrile, Isomer 1 | 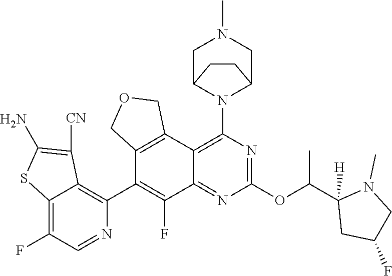 [SMILES: CC(Oc1nc(N2C3CCC2CN(C)C3)c2c3c(c(-c4ncc(F)c5sc(N)c(C#N)c45)c(F)c2n1)COC3)[C@@H]1C[C@@H](F)CN1C] | 651 |
| 5³ | 2-Amino-7-fluoro-4-(5- fluoro-3-(1-((2R,3S)-3- fluoro-1- methylpyrrolidin-2- yl)ethoxy)-1-(3-((R)-2- hydroxypropyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 2 | 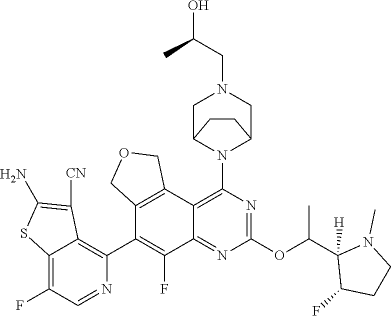 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1[C@@H](F)CCN1C] | 695 |
| 6⁴ | 2-Amino-7-fluoro-4-(5- fluoro-1-(3-((R)-2- hydroxypropyl)-3,8- diazabicyclo[3.2.1]octan- 8-yl)-3-(1-((2S,4R)-4- methoxy-1- methylpyrrolidin-2- yl)ethoxy)-7,9- dihydrofuro[3,4- f]quinazolin-6- yl)thieno[3,2-c]pyridine- 3-carbonitrile, Isomer 1 | 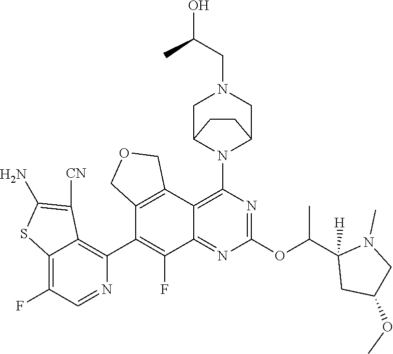 [SMILES: CO[C@@H]1C[C@@H](C(C)Oc2nc(N3C4CCC3CN(C[C@@H](C)O)C4)c3c4c(c(-c5ncc(F)c6sc(N)c(C#N)c56)c(F)c3n2)COC4)N(C)C1] | 707 |
| 7⁴ | 2-Amino-7-fluoro-4-(5- fluoro-3-(1-((2S,4R)-4- methoxy-1- methylpyrrolidin-2- yl)ethoxy)-1-(3-(2- methoxyethyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 1 | 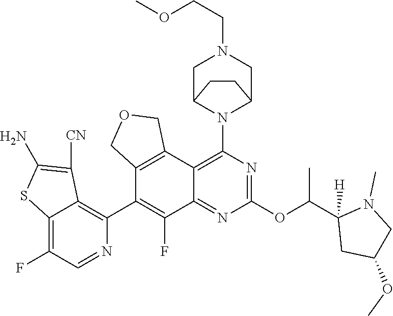 [SMILES: COCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3] | 707 |
| 8⁴ | 2-Amino-7-fluoro-4-(5- fluoro-3-(1-((2S,4R)-4- methoxy-1- methylpyrrolidin-2- yl)ethoxy)-1-(3-(3- methoxypropyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 1 | 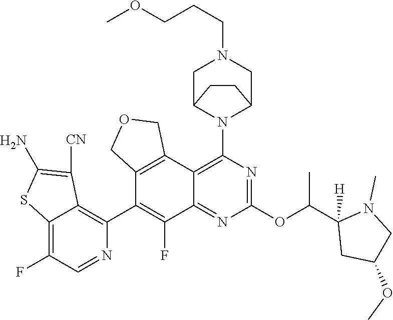 [SMILES: COCCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3] | 721 |
| 9 | 2-Amino-7-fluoro-4-(5- fluoro-1-(3-(3- methoxypropyl)-3,8- diazabicyclo[3.2.1]octan- 8-yl)-3-((S)-1-((S)-1- methypyrrolidin-2- yl)ethoxy)-7,9- dihydrofuro[3,4- f]quinazolin-6- yl)thieno[3,2-c]pyridine- 3-carbonitrile | 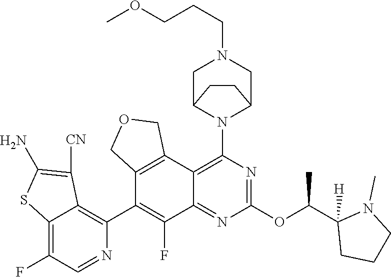 [SMILES: COCCCN1CC2CCC(C1)N2c1nc(O[C@@H](C)[C@@H]2CCCN2C)nc2c(F)c(-c3ncc(F)c4sc(N)c(C#N)c34)c3c(c12)COC3] | 691 |
| 10 | 2-Amino-7-fluoro-4-(5- fluoro-1-(3-methyl-3,8- diazabicyclo[3.2.1]octan- 8-yl)-3-((S)-1-((S)-1- methylpyrrolidin-2- yl)ethoxy)-7,9- dihydrofuro[3,4- f]quinazolin-6- yl)thieno[3,2-c]pyridine- 3-carbonitrile | 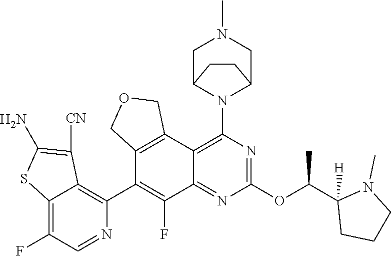 [SMILES: C[C@H](Oc1nc(N2C3CCC2CN(C)C3)c2c3c(c(-c4ncc(F)c5sc(N)c(C#N)c45)c(F)c2n1)COC3)[C@@H]1CCCN1C] | 633 |
| 11 | 2-Amino-7-fluoro-4-(5- fluoro-1-(3-methyl-3,8- diazabicyclo[3.2.1]octan- 8-yl)-3-((R)-1-((S)-1- methylpyrrolidin-2- yl)ethoxy)-7,9- dihydrofuro[3,4- f]quinazolin-6- yl)thieno[3,2-c]pyridine- 3-carbonitrile | 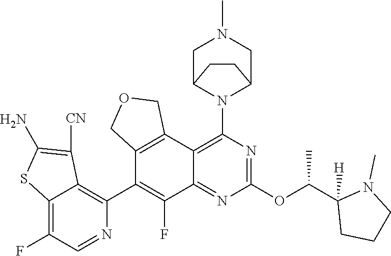 [SMILES: C[C@@H](Oc1nc(N2C3CCC2CN(C)C3)c2c3c(c(-c4ncc(F)c5sc(N)c(C#N)c45)c(F)c2n1)COC3)[C@@H]1CCCN1C] | 633 |

¹Single methyl isomer (from precursor in Preparation 26)
²Single methyl isomer (from precursor in Preparation 27)
³Single methyl isomer (from precursor in Preparation 25)
⁴Single methyl isomer (from precursor in Preparation 28)

The following compounds in Table 10 were prepared in similar manner as described in Preparation 44. Various methods were used to purify the compounds, which would be apparent to one skilled in the art.

TABLE 10

| | | | MS |
|---|---|---|---|
| | | | (ES) m/z |
| Example | Chemical Name | Structure | (M + 1) |
| 12 | 2-Amino-7-fluoro-4-(5- fluoro-1-(3-((R)-2- hydroxypropyl)-3,8- diazabicyclo[3.2.1]octan- 8-yl)-3-((S)-1-((S)-1- methylpyrrolidin-2- yl)ethoxy)-7,9- dihydrofuro[3,4- f]quinazolin-6- yl)thieno[3,2-c]pyridine- 3-carbonitrile | 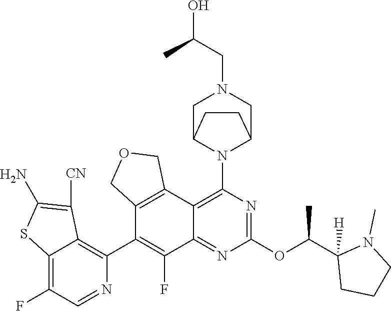 [SMILES: C[C@H](Oc1nc(N2C3CCC2CN(C[C@@H](C)O)C3)c2c3c(c(-c4ncc(F)c5sc(N)c(C#N)c45)c(F)c2n1)COC3)[C@@H]1CCCN1C] | 677 |
| 13 | 2-Amino-7-fluoro-4-(5- fluoro-3-((S)-1-((S)-1- methylpyrrolidin-2- yl)ethoxy)-7,9- dihydrofuro[3,4- f]quinazolin-6- yl)thieno[3,2-c]pyridine- 3-carbonitrile | 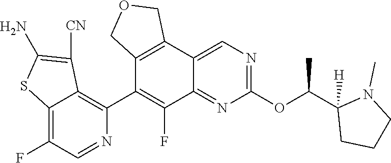 [SMILES: C[C@H](Oc1ncc2c3c(c(-c4ncc(F)c5sc(N)c(C#N)c45)c(F)c2n1)COC3)[C@@H]1CCCN1C] | 509 |
| 14¹ | 2-Amino-7-fluoro-4-(5- fluoro-3-(1-((2S,4R)-4- methoxy-1- methylpyrrolidin-2- yl)ethoxy)-7,9- dihydrofuro[3,4- f]quinazolin-6- yl)thieno[3,2-c]pyridine- 3-carbonitrile, Isomer 1 | 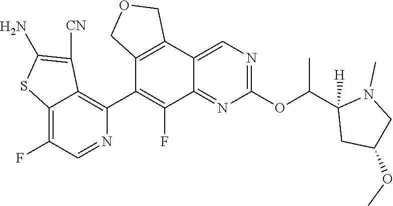 [SMILES: CO[C@@H]1C[C@@H](C(C)Oc2ncc3c4c(c(-c5ncc(F)c6sc(N)c(C#N)c56)c(F)c3n2)COC4)N(C)C1] | 539 |

¹Single methyl isomer (from precursor in Preparation 28)

### Example 15

### 2-Amino-7-fluoro-4-(5-fluoro-1-(3-(3-methoxypropyl)-3,8-diazabicyclo[3.2.1]octan-8-yl)-3-((S)-1-((S)-1-methylpyrrolidin-2-yl)ethoxy)-7,9-dihydrofuro[3,4-f]quinazolin-6-yl)thieno[3,2-c]pyridine-3-carbonitrile methanesulfonic acid

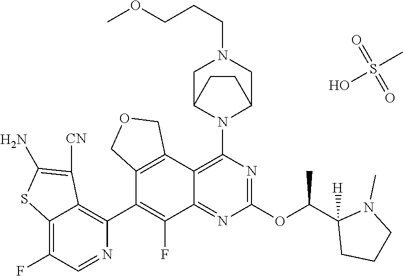

A solution of 2-amino-7-fluoro-4-(5-fluoro-1-(3-(3-methoxypropyl)-3,8-diazabicyclo[3.2.1]octan-8-yl)-3-((S)-1-((S)-1-methylpyrrolidin-2-yl)ethoxy)-7,9-dihydrofuro[3,4-f]quinazolin-6-yl)thieno[3,2-c]pyridine-3-carbonitrile (1.3 g, 1.9 mmol) in DCM (10 mL) and MeOH (10 mL) was treated with methanesulfonic acid (0.13 mL, 1.9 mmol). The reaction mixture was stirred at RT for 20 min, then concentrated under reduced pressure. The solids were placed in a vacuum oven (40° C.) overnight to obtain the title compound (1.5 g, 100%) as a tan solid. MS (ES) m/z=691 (M+1).

The following compounds in Table 11 were prepared in similar manner as described in Example 15. Various methods were used to purify the compounds, which would be apparent to one skilled in the art.

TABLE 11

| | | | MS |
|---|---|---|---|
| | | | (ES) m/z |
| Example | Chemical Name | Structure | (M + 1) |
| 16¹ | 2-Amino-7-fluoro-4-(5- fluoro-3-(1-((2S,4R)-4- fluoro-1- methylpyrrolidin-2- yl)ethoxy)-1-(3-methyl- 3,8- diazabicyclo[3.2.1]octan- 8-yl)-7,9- dihydrofuro[3,4- f]quinazolin-6- yl)thieno[3,2-c]pyridine- 3-carbonitrile, Isomer 1 methanesulfonic acid | 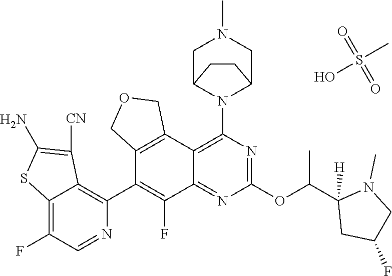 | 651 |
| 17² | 2-Amino-7-fluoro-4-(5- fluoro-1-(3-((R)-2- hydroxypropyl)-3,8- diazabicyclo[3.2.1]octan- 8-yl)-3-(1-((2S,4R)-4- methoxy-1- methylpyrrolidin-2- yl)ethoxy)-7,9- dihydrofuro[3,4- f]quinazolin-6- yl)thieno[3,2-c]pyridine- 3-carbonitrile, Isomer 1 methanesulfonic acid | 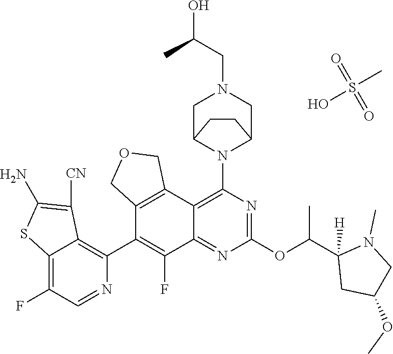 | 707 |
| 18² | 2-Amino-7-fluoro-4-(5- fluoro-3-(1-((2S,4R)-4- methoxy-1- methylpyrrolidin-2- yl)ethoxy)-1-(3-(2- methoxyethyl)-3,8- diazabicyclo[3.2.1]octan- 8-yl)-7,9- dihydrofuro[3,4- f]quinazolin-6- yl)thieno[3,2-c]pyridine- 3-carbonitrile, Isomer 1 methanesulfonic acid | 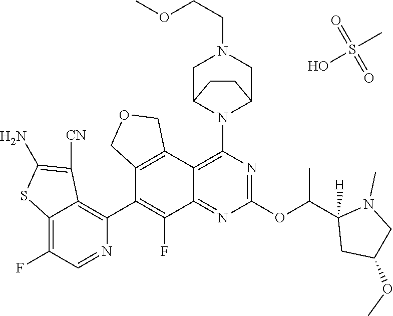 | 707 |
| 19² | 2-Amino-7-fluoro-4-(5- fluoro-3-(1-((2S,4R)-4- methoxy-1- methylpyrrolidin-2- yl)ethoxy)-1-(3-(3- methoxypropyl)-3,8- diazabicyclo[3.2.1]octan- 8-yl)-7,9- dihydrofuro[3,4- f]quinazolin-6- yl)ethieno[3,2-c]pyridine- 3-carbonitrile, Isomer 1 methanesulfonic acid | 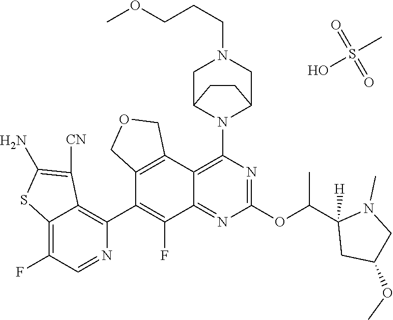 | 721 |
| 20 | 2-Amino-7-fluoro-4-(5- fluoro-1-(3-methyl-3,8- diazabicyclo[3.2.1]octan- 8-yl)-3-((S)-1-((S)-1- methylpyrrolidin-2- yl)ethoxy)-7,9- dihydrofuro[3,4- f]quinazolin-6- yl)thieno[3,2-c]pyridine- 3-carbonitrile methanesulfonic acid | 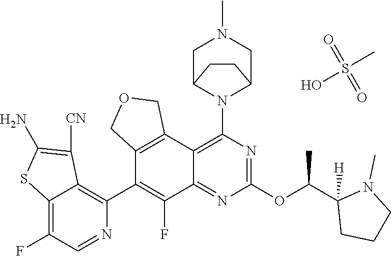 | 633 |

¹Single methyl isomer (from precursor in Preparation 26)
²Single methyl isomer (from precursor in Preparation 28)

### Example 21

### 2-Amino-7-fluoro-4-(5-fluoro-1-(3-(2-methoxyethyl)-3,8-diazabicyclo[3.2.1]octan-8-yl)-3-((S)-1-((S)-1-methylpyrrolidin-2-yl)ethoxy)-7,9-dihydrofuro[3,4-f]quinazolin-6-yl)thieno[3,2-c]pyridine-3-carbonitrile methanesulfonic acid

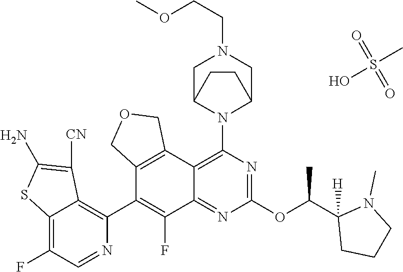

4-(1-(3,8-Diazabicyclo[3.2.1]octan-8-yl)-5-fluoro-3-((S)-1-((S)-1-methylpyrrolidin-2-yl)ethoxy)-7,9-dihydrofuro[3,4-f]quinazolin-6-yl)-2-amino-7-fluorothieno[3,2-c]pyridine-3-carbonitrile and 2-methoxyacetaldehyde were used in a manner analogous to that described for Example 1 to obtain crude 2-amino-7-fluoro-4-(5-fluoro-1-(3-(2-methoxyethyl)-3,8-diazabicyclo[3.2.1]octan-8-yl)-3-((S)-1-((S)-1-methylpyrrolidin-2-yl)ethoxy)-7,9-dihydrofuro[3,4-f]quinazolin-6-yl)thieno[3,2-c]pyridine-3-carbonitrile (1.01 g, 46%) as a tan solid. MS (ES) m/z=677 (M+1).

2-Amino-7-fluoro-4-(5-fluoro-1-(3-(2-methoxyethyl)-3,8-diazabicyclo[3.2.1]octan-8-yl)-3-((S)-1-((S)-1-methylpyrrolidin-2-yl)ethoxy)-7,9-dihydrofuro[3,4-f]quinazolin-6-yl)thieno[3,2-c]pyridine-3-carbonitrile was used in a manner analogous to that described for Example 15 to obtain the title compound (1.11 g, 96%) as a yellow solid. MS (ES) m/z=677 (M+1).

### Biological Assays

The following assays demonstrate that the exemplified compounds are potent inhibitors of Kras G12D and inhibit growth of certain tumors in vitro and/or in vivo.

### PANC-1 Cellular Active RAS GTPase ELISA (KRas G12D Mutation)

The purpose of this assay is to measure the ability of test compounds to inhibit constitutive RAS GTPase activity in human PANC-1 (RRID:CVCL_0480) pancreatic ductal adenocarcinoma cells (Supplier: ATCC #CRL-1469). The RAS GTPase ELISA kit (Active Motif Cat #52097) contains a 96-well glutathione-coated capture plate and kit-supplied Glutathione-S-Transferase (GST)-fused to Raf-Ras Binding Domain (RBD) protein. Activated pan-RAS (GTP-bound) in cell extracts specifically bind to the Raf-RBD. Bound RAS is detected with a primary Ras antibody that recognizes human K-Ras (and H-Ras). An HRP-conjugated anti-rat IgG secondary antibody recognizes the primary antibody, and a development substrate solution facilitates a chemiluminescent readout.

PANC-1 cells are plated at a concentration of 75,000 cells/well in 80 μL complete media (DMEM, high-glucose, L-glutamine, GIBCO; 10% heat-inactivated fetal bovine serum, GIBCO) and incubated overnight at 37° C./5% CO₂. Approximately 24 hours later, 20 μL of (1:3) serially-diluted (in complete media) test compound (1-50 μM top concentration) and 20 μL of serially-diluted (in complete media) controls (Maximum signal wells: 0.5% DMSO and Minimum signal wells: 10 μM reference positive control compound) are added to the cell plate and incubated for 2 hours at 37° C./5% CO₂. Complete Lysis/Binding Buffer is prepared containing Protease Inhibitor cocktail (PIC) and stored on ice. One hour before cell plate incubation is completed, GST-Raf-RBD is diluted in lysis/binding buffer, and 50 μL of mixed buffer per well is added to the supplied opaque white ELISA assay plate and is incubated for a minimum of 1 hour at 4° C., with gently rocking. After 2 hours, the cells are washed with 100 μL ice-cold Ca2+/Mg2+-free PBS and lysed with 100 μL of kit supplied lysis/binding buffer (AM11). After 30-50 minutes of vigorous plate shaking at ambient temperature, cell plate is centrifuged at 410×g (approx. 1500 rpm) for 10 minutes. Wash buffer diluted to 1× with ultrapure 120 and 0.2 μm filtered is prepared at ambient temperature during the centrifugation step and then used to wash (3×100 μL) the GST-Raf-RBD coated assay plate. Next, 50 μL of cell lysate is added to the GST-Raf-RBD coated assay plate and incubated for 1 hour at ambient temperature with gentle shaking. During this incubation period, 1× Antibody Binding Buffer is prepared from thawed concentrate. The assay plate is washed 3×100 μL with 1× Wash Buffer, and then 50 μL of Primary RAS Antibody (kit supplied #101678), diluted 1:500 in 1× Antibody Binding buffer, is added. After a one hour of ambient incubation with gentle shaking, the assay plate is washed 3×100 μL with 1× Wash Buffer. Subsequently, 50 μL of Anti-rat HRP-conjugated IgG secondary antibody (0.25 μg/μL) (diluted 1:5000 in 1× Antibody Binding buffer) is added to each well of the assay plate and incubated an additional hour at ambient temperature with gentle shaking. Finally, the assay plate is washed 4×100 μL with 1× Wash buffer, followed by addition of 50 μL of mixed ambient temperature chemiluminescent working solution (combination of Reaction buffer with a chemiluminescence substrate). Data from each well's luminescent emission is recorded with a 2104 EnVision™ Plate Reader (Perkin Elmer) using a luminescence program optimized for the assay plate dimensions.

The signal is converted to percent inhibition using the following equation: % Inhibition=100−[(Test Compound Signal−Median Minimum Signal)/(Median Maximum Signal−Median Minimum Signal)×100]. The Maximum signal is a control well without inhibitor (DMSO). The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®, v17: y=(A+((B−A)/(1+((x/C)\hat{ }D)))) where, y=% inhibition, A=minimum asymptote, B=maximum asymptote, C=relative IC₅₀ or the inhibitor concentration producing 50% inhibition within the fitted range of both asymptotes, and D=Hill Slope.

In the above assay, compounds of Examples 1, 2, 4, 6-12, and 15-21 were tested and all exhibited an ability to inhibit constitutive RAS GTPase activity indicating inhibition of KRas G12D mutant enzyme with a relative IC₅₀ of <100 nM. This data shows that compounds of Formula I as described herein are potent inhibitors of KRAS-GTP activity in this human pancreatic cancer cell culture demonstrating the ability to inhibit KRas G12D mutants.

### MKN-45 Cellular Active RAS GTPase ELISA (KRas Wild-Type)

The purpose of this assay is to measure the ability of test compounds to inhibit constitutive RAS GTPase activity in human MKN-45 gastric adenocarcinoma cell (Supplier: JCRB, SupplierID: JCRB 0254, Lot: 05222009). The RAS GTPase ELISA kit (Active Motif Cat #52097) contains a 96-well glutathione-coated capture plate and kit-supplied Glutathione-S-Transferase (GST)-fused to Raf-Ras Binding Domain (RBD) protein. Activated pan-RAS (GTP-bound) in cell extracts specifically bind to the Raf-RBD. Bound RAS is detected with a primary Ras antibody that recognizes human K-Ras (and H-Ras). An HRP-conjugated anti-rat IgG secondary antibody recognizes the primary antibody, and a development substrate solution facilitates a chemiluminescent readout.

MKN-45 cells are plated at a concentration of 75,000 cells/well in 80 μL complete media (DMEM, high-glucose, L-glutamine, GIBCO; 10% heat-inactivated fetal bovine serum, GIBCO) and incubated overnight at 37° C./5% CO₂. Approximately 24 hours later, 20 μL of (1:3) serially-diluted (in complete media) test compound (1-10 μM top concentration) and 20 μL of serially-diluted (in complete media) controls (Maximum signal wells: 0.1% DMSO and Minimum signal wells: 10 μM reference positive control compound) are added to the cell plate and incubated for 2 hours at 37° C./5% CO₂. Complete Lysis/Binding Buffer is prepared containing Protease Inhibitor cocktail (PIC) and stored on ice. One hour before cell plate incubation is completed, GST-Raf-RBD is diluted in lysis/binding buffer, and 50 μL of mixed buffer per well is added to the supplied opaque white ELISA assay plate and is incubated for a minimum of 1 hour at 4° C., with gently rocking. After 2 hours, the cells are washed with 100 μL ice-cold Ca2+/Mg2+-free PBS and lysed with 100 μL of kit supplied lysis/binding buffer (AM11). After 30-50 minutes of vigorous plate shaking at ambient temperature, cell plate is centrifuged at 410×g (approx. 1500 rpm) for 10 minutes. Wash buffer diluted to 1× with ultrapure H₂O during the centrifugation step and then used to wash (3×100 μL) the GST-Raf-RBD coated assay plate. Next, 50 μL of cell lysate is added to the GST-Raf-RBD coated assay plate and incubated for 1 hour at ambient temperature with gentle shaking. During this incubation period, 1× Antibody Binding Buffer is prepared from thawed concentrate. The assay plate is washed 3×100 μL with 1× Wash Buffer, and then 50 μL of Primary RAS Antibody (kit supplied #101678), diluted 1:500 in 1× Antibody Binding buffer, is added. After a one hour of ambient incubation with gentle shaking, the assay plate is washed 3×100 μL with 1× Wash Buffer. Subsequently, 50 μL of Anti-rat HRP-conjugated IgG secondary antibody (0.25 μg/μL) (diluted 1:5000 in 1× Antibody Binding buffer) is added to each well of the assay plate and incubated an additional hour at ambient temperature with gentle shaking. Finally, the assay plate is washed 4×100 μL with 1× Wash buffer, followed by addition of 50 μL of mixed ambient temperature chemiluminescent working solution (combination of Reaction buffer with a chemiluminescence substrate). Data from each well's luminescent emission is recorded with a 2104 EnVision™ Plate Reader (Perkin Elmer) using a luminescence program optimized for the assay plate dimensions.

The signal is converted to percent inhibition using the following equation: % Inhibition=100−[(Test Compound Signal−Median Minimum Signal)/(Median Maximum Signal−Median Minimum Signal)×100]. The Maximum signal is a control well without inhibitor (DMSO). The Minimum signal is a control well containing a reference inhibitor sufficient to fully inhibit activity. The IC₅₀ is determined by fitting the percent inhibition at each inhibitor concentration to the four parameter nonlinear logistic equation using Genedata Screener®, v17: y=(A+((B−A)/(1+((x/C)\hat{ }D)))) where, y=% inhibition, A=minimum asymptote, B=maximum asymptote, C=relative IC₅₀ or the inhibitor concentration producing 50% inhibition within the fitted range of both asymptotes, and D=Hill Slope.

Compounds of Examples 1, 2, 4, 6-8, 10-12, and 15-21 were tested in both assays above (PANC-1 Cellular Active RAS GTPase ELISA and MKN-45 Cellular Active RAS GTPase ELISA) and all showed a significant (i.e., greater than 10-fold) selective inhibition preference for KRas G12D mutant over KRas wild-type.

### Cellular Phospho-ERK AlphaLISA® Assay for KRAS Inhibition

The purpose of these assays is to quantify the ability of test compounds to selectively inhibit KRAS signaling in cells with amplified KRAS and expressing activating KRAS G12D mutation (Table 12). Cancer cell lines used in this study were selected based on the presence of homozygous activating KRAS G12D mutation, or amplification of the KRAS gene.

TABLE 12
Cell Line Information

| Cell Line | | Assay Seeding |
|---|---|---|
| Name | RAS Mutation/Features | Density (Cells/Well) |
| HPAC | KRAS G12D/Human Pancreatic Cancer | 2, 500 |

The compounds' activity is determined by measuring changes in the phosphorylation levels of the downstream effector Extracellular Signal-regulated Kinase-1 and 2 (ERK1/2) in the compound treated cells. Phosphorylation levels of ERK-1/2 are measured using the AlphaLISA® SureFire® Ultra™ p-ERK 1/2 (Thr202/Tyr204) Assay Kit (#ALSU-PERK-A50K, PerkinElmer® Waltham, MA). The AlphaLISA® assay is a quantitative sandwich immunoassay that can be used to detect phosphorylation of target proteins from cellular lysates using bead-based Alpha technology. The assay kit contains two antibodies, one that binds the phospho-Thr202/Tyr204 epitope on ERK-1/2, and another one that recognizes a separate site on the protein. One of these antibodies is biotinylated and associated with streptavidin-coated Alpha Donor beads, the other antibody is conjugated to AlphaLISA® Acceptor beads. When ERK-1/2 is phosphorylated in cellular lysate, the Donor and Acceptor beads are brought into proximity with each other. When the Donor bead is excited by 600 nm wavelength light, a photosensitizer inside the bead converts ambient oxygen to an excited singlet state. When the Acceptor bead is within 200 nm of this reaction, the singlet oxygen reacts with the Acceptor leading to a chemiluminescent emission. The amount of light measured is proportional to the amount of phosphorylated ERK-1/2 in the lysate. The AlphaLISA® SureFire® Ultra™ p-ERK 1/2 (Thr202/Tyr204) Assay Kit contains AlphaLISA® antibody-conjugated Donor and Acceptor Beads, Lysis buffer concentrate, and a set of proprietary buffers (Activation Buffer, Reaction Buffer 1, Reaction Buffer 2, and Dilution Buffer).

To perform the assays, test compounds and controls are acoustically dispensed (Labcyte ECHO®, San Jose, CA) into a white 384-well assay plate (Proxiplate-384, PerkinElmer #6008280) in a 10-point 3-fold dilution series in 30 nL DMSO. Cells are then added to the assay plate in 8 μL per well assay medium (HBSS, Sigma #55021C, 10% FBS, GIBCO #10082-147) at a cell line specific density (Table 18). The final compound concentrations range from 0.5 to 10,000 nM and the final DMSO concentration is 0.375% in each well. Maximum signal control wells contain 0.375% DMSO only (negative control), and minimum signal control wells contain 10,000 nM control compound (positive control). Cells in suspension are incubated with the test and reference compounds for 2 h at 37° C./5% CO₂. Following the 2 h incubation, cells are lysed by adding 2 μL of the AlphaLISA® Lysis buffer concentrate (5×) supplemented with protease/phosphatase inhibitor cocktail (Thermo Scientific #78442). The assay plate is covered with an opaque lid and shaken at 750 rpm on a multi-plate shaker (Heidolph, Schwabach, Germany) for 30 min at room temperature to induce cell lysis. During the lysis, the AlphaLISA® Acceptor beads are diluted 1:50 in a prepared buffer mixture (1:1 AlphaLISA® Reaction Buffers 1 and 2 with a 1:25 dilution of AlphaLISA® Activation Buffer). Following cell lysis, plates are centrifuged briefly, and 5 μL per well prepared Acceptor beads are added. The plate is then covered and incubated in the dark for 2 h at room temperature. During the Acceptor bead incubation, Donor beads are prepared by diluting the Alpha streptavidin Donor beads 1:50 in AlphaLISA® Dilution buffer. Following the Acceptor bead incubation, 5 μL per well of Donor bead mixture is added to the plates. Plates are then covered and allowed to incubate in the dark at room temperature for 2 h. After this incubation period, the AlphaLISA signal is read using a PHERAstar® FSX multimode plate reader (BMG Labtech, Ortenberg, Germany) equipped with an AlphaLISA® compatible optics cube.

Raw signal obtained from the AlphaLISA® assay is analyzed using Genedata Screener® 17.0.3. Within the program, data is normalized to 32 wells treated with inhibition control (max inhibition/positive control) and 32 wells treated with 0.375% DMSO only (minimum inhibition/negative control) to calculate the % Activity of the compound:

$$\% Activity = 100 \times ( 1 - \frac{( treated value - positive control )}{( negative control - positive control )} ) \qquad eq . 1$$

% Activity values are fit to a four-parameter non-linear logistic equation using Genedata Screener® 17.0.3. to-determine IC₅₀ values:

$$y = Bottom + \frac{Top - Bottom}{1 + {( \frac{{10}^{Log ( {IC}_{50} )}}{{10}^{x}} )}^{h}} \qquad eq . 2$$

Where y=% Activity, Bottom=minimum asymptote, Top=maximum asymptote, x=compound concentration, IC₅₀=the compound concentration where half maximal activity is achieved, and h=the Hill Coefficient.

In the above assays, compounds of Examples 13-21 were tested and exhibited an ability to reduce levels of phosphorylated ERK-1/2 in cells expressing KRAS G12D indicating inhibition of constitutive RAS activity in cells expressing KRAS G12D with a relative IC50 of <100 nM. This data shows that compounds of Formula I as described herein are potent inhibitors of KRAS human cancer cells expressing KRAS G12D demonstrating the ability to inhibit KRAS signaling in these cells.

TABLE 13
Abbreviations

| | |
|---|---|
| KRAS | Kirsten Rat Sarcoma Virus |
| MEF | Mouse Embryonic Fibroblasts |
| ERK | Extracellular Signal-Regulated Kinase |
| AlphaLISA | Alpha-Linked Immunosorbent Assay |
| DMSO | Dimethyl Sulfoxide |
| HBSS | Hank's Balanced Salt Solution |
| FBS | Fetal Bovine Serum |
| CO₂ | Carbon Dioxide |

What is claimed is:

1. A compound of the formula: 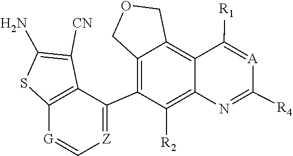
wherein
A is —C(H)— or —N—;
Z is —C(R_{3c})— or —N—;
G is —C(R_{3b})— or —N—;
R₁ is —H, or a group of the formula 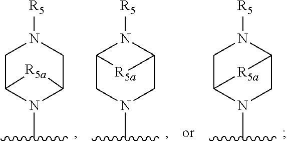
R₂ is —H, halogen, or methyl;
R_{3b}, and R_{3c} are each independently —H, halogen, or methyl;
R₄ is a group of the formula 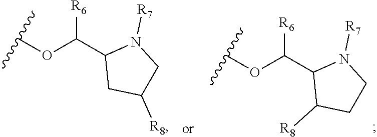
R₅ is a C₁₋₄ alkyl optionally substituted with one or more hydroxyl, or methoxy;
R₅ₐ is C₁₋₃ alkylene;
R₆ is C₁₋₃ alkyl;
R₇ is —H, or C₁₋₃ alkyl; and
R₈ is —H, halogen, or C₁₋₃ alkoxy; or a pharmaceutically acceptable salt thereof.

2. The compound according to claim 1, wherein G is —N—, or a pharmaceutically acceptable salt thereof.

3. The compound according to claim 1, wherein G is —C(R_{3b})—, or a pharmaceutically acceptable salt thereof.

4. The compound according to claim 3, wherein R_{3b} is —F, or a pharmaceutically acceptable salt thereof.

5. The compound according to claim 1, wherein Z is —N—, or a pharmaceutically acceptable salt thereof.

6. The compound according to claim 1, wherein Z is —C(R_{3c})—, or a pharmaceutically acceptable salt thereof.

7. The compound according to claim 6, wherein R_{3c} is —H, or —F, or a pharmaceutically acceptable salt thereof.

8. The compound according to claim 6, wherein R_{3c} is —F, or a pharmaceutically acceptable salt thereof.

9. The compound according to claim 1, wherein A is —N—, or a pharmaceutically acceptable salt thereof.

10. The compound according to claim 1, wherein A is —C(H)—, or a pharmaceutically acceptable salt thereof.

11. The compound according to claim 1, wherein R₂ is F or Cl, or a pharmaceutically acceptable salt thereof.

12. The compound according to claim 1, wherein R₂ is —F, or a pharmaceutically acceptable salt thereof.

13. The compound according to claim 1, wherein R₂ is Cl, or a pharmaceutically acceptable salt thereof.

14. The compound according to claim 1, wherein R₁ is —H, or a pharmaceutically acceptable salt thereof.

15. The compound according to claim 1, wherein R₁ is a group of the formula 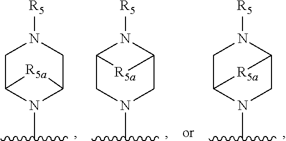
or a pharmaceutically acceptable salt thereof.

16. The compound according to claim 15, wherein R₁ is a group of the formula 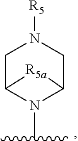
or a pharmaceutically acceptable salt thereof.

17. The compound according to claim 16, wherein R₅ₐ is ethylene, or a pharmaceutically acceptable salt thereof.

18. The compound according to claim 17, wherein R₁ is selected from 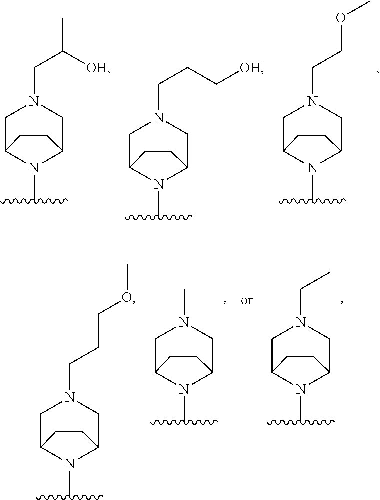
or a pharmaceutically acceptable salt thereof.

19. The compound according to claim 18, wherein R₁ is selected from 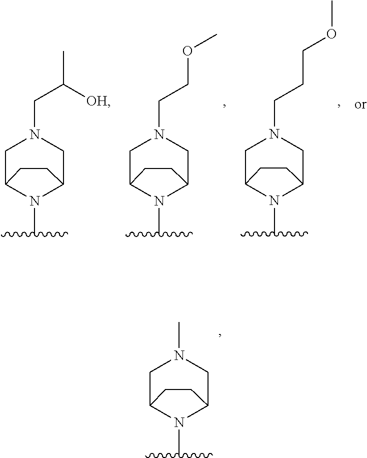
or a pharmaceutically acceptable salt thereof.

20. The compound according to claim 1, wherein R₄ is a group of the formula 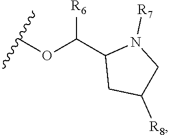
or a pharmaceutically acceptable salt thereof.

21. The compound according to claim 20, wherein R₄ is a group of the formula 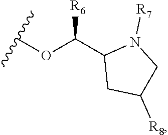
or a pharmaceutically acceptable salt thereof.

22. The compound according to claim 20, wherein R₄ is a group of the formula 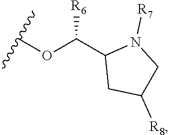
or a pharmaceutically acceptable salt thereof.

23. The compound according to claim 1, wherein R₄ is a group of the formula 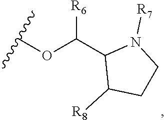
or a pharmaceutically acceptable salt thereof.

24. The compound according to claim 23, wherein R₄ is a group of the formula 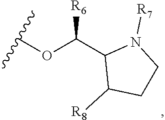
or a pharmaceutically acceptable salt thereof.

25. The compound according to claim 23, wherein R₄ is a group of the formula 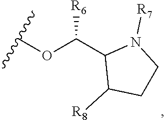
or a pharmaceutically acceptable salt thereof.

26. The compound according to claim 1, wherein R₆ is methyl, or a pharmaceutically acceptable salt thereof.

27. The compound according to claim 1, wherein R₇ is methyl, or a pharmaceutically acceptable salt thereof.

28. The compound according to claim 1, wherein R₈ is —H, —F, or methoxy, or a pharmaceutically acceptable salt thereof.

29. The compound according to claim 28, wherein R₈ is —H, or a pharmaceutically acceptable salt thereof.

30. The compound according to claim 28, wherein R₈ is —F, or a pharmaceutically acceptable salt thereof.

31. The compound according to claim 28, wherein R₈ is methoxy, or a pharmaceutically acceptable salt thereof.

32. The compound according to claim 1, wherein R₄ is selected from 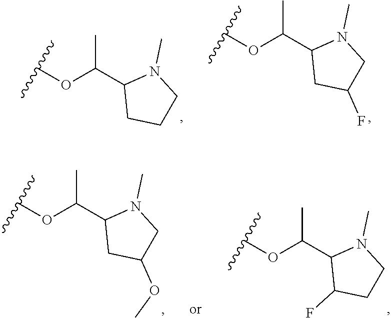
or a pharmaceutically acceptable salt thereof.

33. The compound according to claim 32, wherein R₄ is selected from 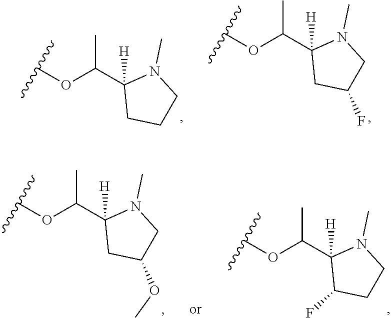
or a pharmaceutically acceptable salt thereof.

34. The compound according to claim 32, wherein R₄ is selected from 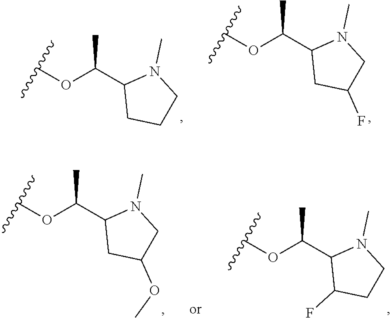
or a pharmaceutically acceptable salt thereof.

35. The compound according to claim 32, wherein R₄ is selected from 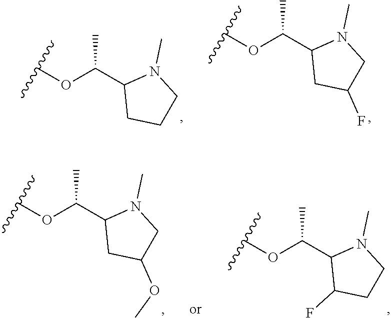
or a pharmaceutically acceptable salt thereof.

36. The compound according to claim 34, wherein R₄ is selected from 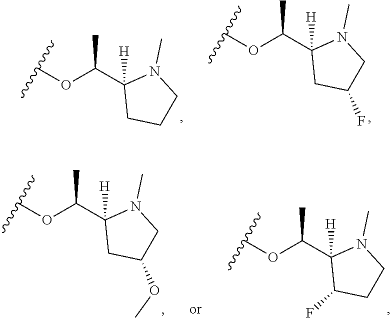
or a pharmaceutically acceptable salt thereof.

37. The compound according to claim 1, selected from 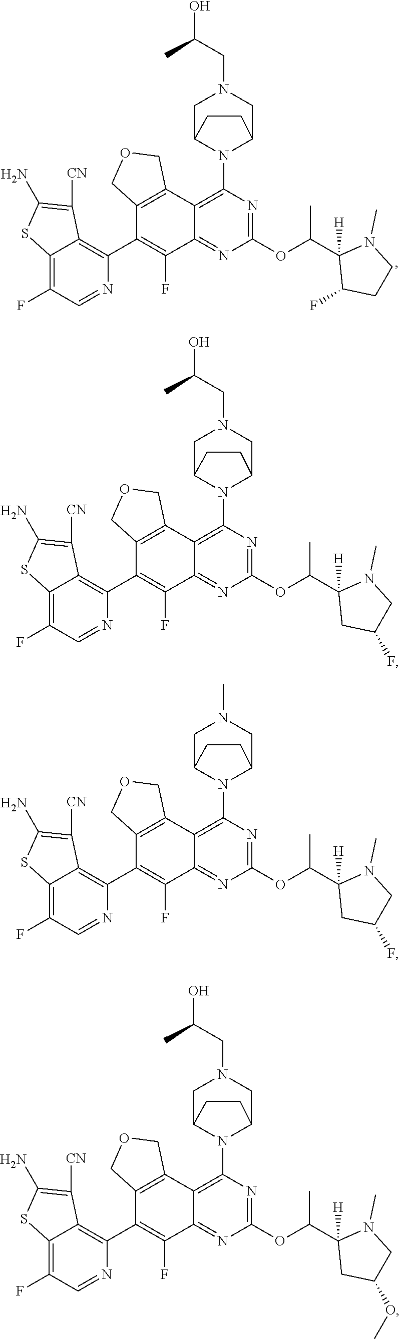 [SMILES: CC(Oc1nc(N2C3CCC2CN(C[C@@H](C)O)C3)c2c3c(c(-c4ncc(F)c5sc(N)c(C#N)c45)c(F)c2n1)COC3)[C@@H]1[C@@H](F)CCN1C ; CC(Oc1nc(N2C3CCC2CN(C[C@@H](C)O)C3)c2c3c(c(-c4ncc(F)c5sc(N)c(C#N)c45)c(F)c2n1)COC3)[C@@H]1C[C@@H](F)CN1C ; CC(Oc1nc(N2C3CCC2CN(C)C3)c2c3c(c(-c4ncc(F)c5sc(N)c(C#N)c45)c(F)c2n1)COC3)[C@@H]1C[C@@H](F)CN1C ; CO[C@@H]1C[C@@H](C(C)Oc2nc(N3C4CCC3CN(C[C@@H](C)O)C4)c3c4c(c(-c5ncc(F)c6sc(N)c(C#N)c56)c(F)c3n2)COC4)N(C)C1] 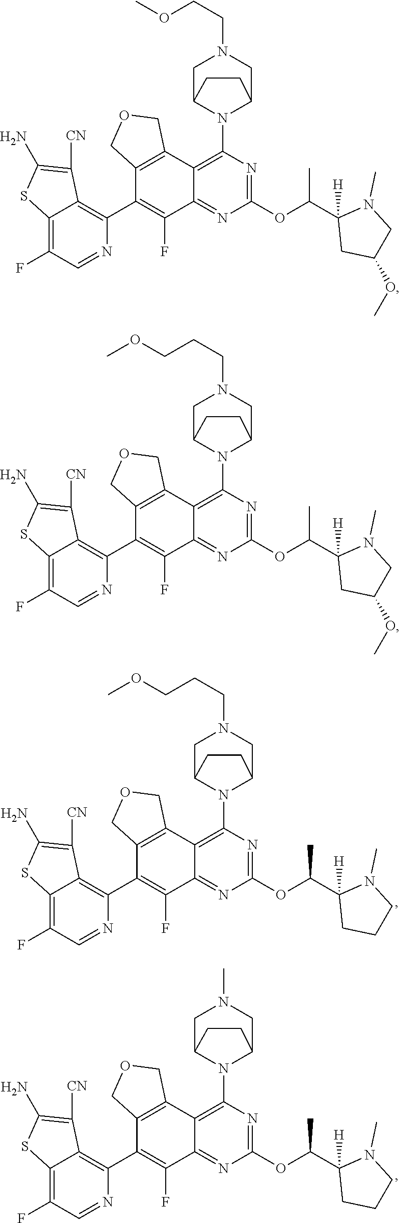 [SMILES: COCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3 ; COCCCN1CC2CCC(C1)N2c1nc(OC(C)[C@@H]2C[C@@H](OC)CN2C)nc2c(F)c(-c3ncc(F)c4sc(N)c(C#N)c34)c3c(c12)COC3 ; COCCCN1CC2CCC(C1)N2c1nc(O[C@@H](C)[C@@H]2CCCN2C)nc2c(F)c(-c3ncc(F)c4sc(N)c(C#N)c34)c3c(c12)COC3 ; C[C@H](Oc1nc(N2C3CCC2CN(C)C3)c2c3c(c(-c4ncc(F)c5sc(N)c(C#N)c45)c(F)c2n1)COC3)[C@@H]1CCCN1C] 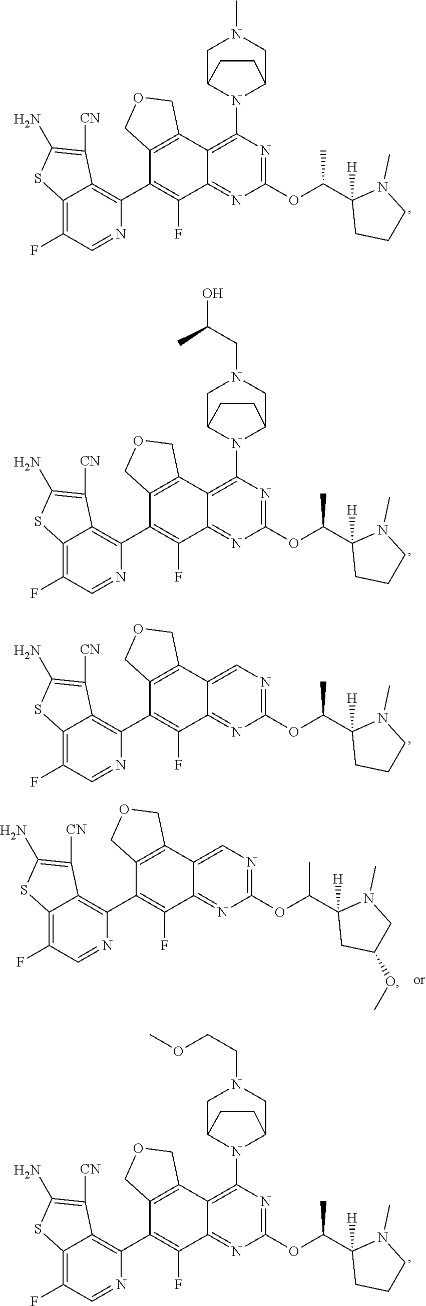
or a pharmaceutically acceptable salt thereof.

38. A pharmaceutical composition comprising a compound according to claim 1, or a pharmaceutically acceptable salt thereof, and a pharmaceutically acceptable carrier, diluent or excipient.

39. A method of treating a patient for cancer, comprising administering to a patient in need thereof, an effective amount of a pharmaceutical composition according to claim 38, wherein the cancer is selected from lung cancer, pancreatic cancer, cervical cancer, esophageal cancer, endometrial cancer, ovarian cancer, cholangiocarcinoma, and colorectal cancer.

40. A method of treating a patient for cancer, comprising administering to a patient in need thereof, an effective amount of a compound according to claim 1, or a pharmaceutically acceptable salt thereof, wherein the cancer is selected from lung cancer, pancreatic cancer, cervical cancer, esophageal cancer, endometrial cancer, ovarian cancer, cholangiocarcinoma, and colorectal cancer.

41. The method according to claim 40 wherein the patient has a cancer that was determined to have one or more cells expressing the KRas G12D mutant protein prior to administration of the compound or a pharmaceutically acceptable salt thereof.

42. The method according to claim 39, wherein the cancer is non-small cell lung cancer.

43. The method according to claim 39, wherein the cancer is colorectal cancer.

44. The method according to claim 39, wherein the cancer is pancreatic cancer.

45. The method according to claim 39, wherein one or more cells express KRas G12D mutant protein.

46. A method of treating a patient with a cancer that has a KRas G12D mutation comprising administering to a patient in need thereof an effective amount of a compound according to claim 1, or a pharmaceutically acceptable salt thereof.

47. The method according to claim 46, wherein the cancer is selected from lung cancer, pancreatic cancer, cervical cancer, esophageal cancer, endometrial cancer, mutant ovarian cancer, cholangiocarcinoma, and colorectal cancer.

48. The method according to claim 47, wherein the cancer is non-small cell lung cancer.

49. The method according to claim 47, wherein the cancer is colorectal cancer.

50. The method according to claim 47, wherein the cancer is pancreatic cancer.

51. The method according to claim 39, wherein the patient is also administered an effective amount of one or more of a PD-1 inhibitor, a PD-L1 inhibitor, a CDK4/CDK6 inhibitor, an EGFR inhibitor, an ERK inhibitor, an Aurora A inhibitor, a SHP2 inhibitor, a platinum agent, and pemetrexed, or pharmaceutically acceptable salts thereof.
